(19) United States
(12) Patent Application Publication
(10) Pub. No.: US 2006/0094749 A1
(43) Pub. Date: May 4, 2006

(54) Substituted pyridoxines as anti-platelet agents

(75) Inventors: Wasimul Haque, Edmonton (CA); James Diakur, Winnipeg (CA); Vinh Pham, Winnipeg (CA); Wenlian Zhang, Guelph (CA)

Correspondence Address: MERCHANT & GOULD PC, P.O. BOX 2903, MINNEAPOLIS, MN, 55402-0903, US

(73) Assignee: Medicure International Inc.

(21) Appl. No.: 10/974,718

(22) Filed: Oct. 28, 2004

(51) Int. Cl. C07D 491/02; C07D 213/63; A61K 31/44; A61K 31/4741

(52) U.S. Cl. 514/302; 514/346; 514/347; 546/115; 546/291

(57) ABSTRACT

Compounds with antiplatelet aggregation characteristics for the treatment of cardiovascular and cardiovascular related disease, are described. The methods are directed to administering pharmaceutical compositions comprising a pyridoxine analogue.

### FIELD OF THE INVENTION

This invention relates to pyridoxine analogues and methods of treating cardiovascular and cardiovascular related diseases by administering pharmaceutical compositions comprising a pyridoxine analogue.

### BACKGROUND

Thrombosis, the development of blood clots within arterial vessels, is due to a complex mechanism involving the activation of both platelet aggregation and the coagulation protease cascade (Ann. Intern Med. (2001) 134: 224-38; N. Engl. J. Med. (2002) 347: 5-12; Thromb. Haemost. (2002) 86: 51-6). The pathways involved normally inhibit blood loss after vessel injury, but in thrombosis and related conditions, these reactions are inappropriately initiated and propagated.

On the molecular level, thrombosis is initiated by the release of mediators such as tissue factor (TF), von Willebrand Factor (vWF) (J. Thromb. Haemost. (2003) 1: 1602-12), and collagen from ruptured atherosclerotic plaques or from damaged blood vessels. Collagen and vWF bind to receptors on platelets and initiate their activation. Once activated, platelets release secretory granules containing ADP, ATP, and calcium (Curr. Opin. Hematol. (2001) δ: 270-6). Activated platelets also synthesize and release thromboxane. The released ADP and thromboxane bind to receptors on the platelets to further propagate platelet activation. Once platelets are activated they start aggregating to initiate clot formation.

TF and vWF also initiate the blood coagulation cascade, which consists of two separate pathways that converge on a common endpoint. Both pathways involve the serial activation of the serine protease clotting factors and ultimately lead to the activation of thrombin. Thrombin, once activated, cleaves fibrinogen to form fibrin. Thrombin, Factor Xa, and Factor VIIa can also activate platelets by cleaving the G protein-coupled protease-activated receptors PAR-1, PAR-3, and PAR-4 (Chest (2003) 124: 18S-25S). PAR-1, the prototype receptor, is activated following cleavage of its amino-terminal exodomain to produce a new amino-terminus (Cell (1991) 64: 1057-68). The new amino terminus then binds to the receptor to effect signaling (J. Biol. Chem. (1994) 269: 16041-45). PARs are therefore peptide receptors that contain their own ligand. PAR-2 is activated by trypsin and not by thrombin (Proc. Natl. Acad. Sci. USA (1994) 91: 9208-12).

Therefore, there is a need for compounds that inhibit the proteases of the blood and thus block platelet aggregation.

### SUMMARY OF THE INVENTION

One embodiment of the invention includes substituted pyridoxine analogues, compositions containing the pyridoxine analogues, and methods of treatment using therapeutically effective amounts of pyridoxine analogues. Compounds and compositions of the invention can be used to treat cardiovascular, cerebrovascular or related diseases and symptoms thereof.

The invention provides compounds of the formula I: 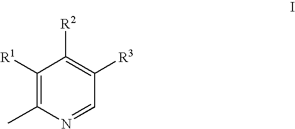
wherein
R¹ is OH, O-alkyl, or O-alkyl-aryl-R⁴, where R⁴ is H, —CN, amidine, alkyl, or cycloalkyl;
R² is alkyl; —(CH₂)_{n′}OH where n′ is an integer from 1 to 8; —(CH₂)ₙCOOH where n is an integer from 0 to 8; —(CH₂)ₙCOO(CH₂)ₙCH₃ where n is as defined above; (CH₂)ₙ-aryl-R⁵ where n is as defined above, and R⁵ is —CN or amidine; (CH₂)ₙ-aryl-aryl-R⁵, where n and R⁵ are as defined above; (CH₂)ₙ—NH-aryl-R⁵, where n and R⁵ are as defined above; (CH₂)ₙ—NH—CO-aryl-R⁴ where n and R⁴ are as defined above; (CH₂)ₙ—NH-aryl-aryl-R⁵ where n and R⁵ are as defined above; and (CH₂)ₙ—NH—CO-aryl-aryl-R⁶ where n is as defined above and R⁶ is —CN, —NO₂, NH₂, or amidine; and
R³ is —(CH₂)_{n′}OH where n′ is as defined above; (CH₂)ₙ—NH-aryl-R⁵, where n and R⁵ are as defined above; (CH₂)ₙ—NH—CO-aryl-R⁴ where n and R⁴ are as defined above; (CH₂)ₙ—NH-aryl-aryl-R⁵ where n and R⁵ are as defined above; and (CH₂)ₙ—NH—CO-aryl-aryl-R⁶ where n and R⁶ are as defined above;
R¹ and R² when taken together form compounds of formula II 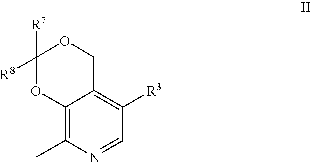
wherein R³ is as defined above;
R⁷ and R⁸ can independently be H or CH₃;
with the proviso that R³ is not CH₂—NH-Phenyl-R⁵ or CH₂—NH-Phenyl-Phenyl-R⁵; and
wherein only one of R⁴, R⁵, and R⁶ can be amidine; or pharmaceutically acceptable salts thereof.

### DETAILED DESCRIPTION OF THE INVENTION

The invention provides compounds of the formula I: 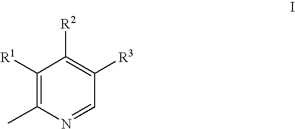
wherein
R¹ is OH, O-alkyl, or O-alkyl-aryl-R⁴, where R⁴ is H, —CN, amidine, alkyl, or cycloalkyl;
R² is alkyl; —(CH₂)_{n′}OH where n′ is an integer from 1 to 8; —(CH₂)ₙCOOH where n is an integer from 0 to 8; —(CH₂)ₙCOO(CH₂)ₙCH₃ where n is as defined above; (CH₂)ₙ-aryl-R⁵ where n is as defined above, and R⁵ is —CN or amidine; (CH₂)ₙ-aryl-aryl-R⁵, where n and R⁵ are as defined above; (CH₂)ₙ—NH-aryl-R⁵, where n and R⁵ are as defined above; (CH₂)ₙ—NH—CO-aryl-R⁴ where n and R⁴ are as defined above; (CH₂)ₙ—NH-aryl-aryl-R⁵ where n and R⁵ are as defined above; and (CH₂)ₙ—NH—CO-aryl-aryl-R⁶ where n is as defined above and R⁶ is —CN, —NO₂, NH₂, or amidine; and
R³ is —(CH₂)_{n′}OH where n′ is as defined above; (CH₂)ₙ—NH-aryl-R⁵, where n and R⁵ are as defined above; (CH₂)ₙ—NH—CO-aryl-R⁴ where n and R⁴ are as defined above; (CH₂)ₙ—NH-aryl-aryl-R⁵ where n and R⁵ are as defined above; and (CH₂)ₙ—NH—CO-aryl-aryl-R⁶ where n and R⁶ are as defined above; R¹ and R² when taken together form compounds of formula II 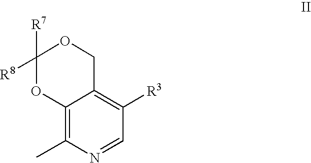
wherein R³ is as defined above;
R⁷ and R⁸ can independently be H or CH₃;
with the proviso that R³ is not CH₂—NH-Phenyl-R⁵ nor CH₂—NH-Phenyl-Phenyl-R⁵; and
wherein only one of R⁴, R⁵, and R⁶ can be amidine; or pharmaceutically acceptable salts thereof.

The invention also provides compounds of formula III. 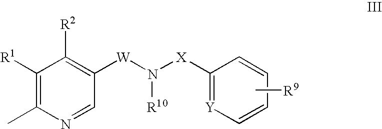
wherein
R¹ is OH, OCH₃, or OCH₂-(4-tert-butylphenyl);
R² is CH₂OH, CH₂OCH₃, CH₂OBn, CH₃, 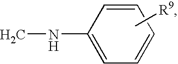
or COOR¹¹ where R¹¹ is H or alkyl;
W is (CH₂)_{n′} where n′=1, 2 or 3, or C═O;
X is (CH₂)ₙ where n=0, 1, 2, or 3, C═O, or CHCH₂CO₂H;
Y is C—H, C—F, C—OCH₃, C—OCF₃, C—CF₃, or N;
R⁹ is 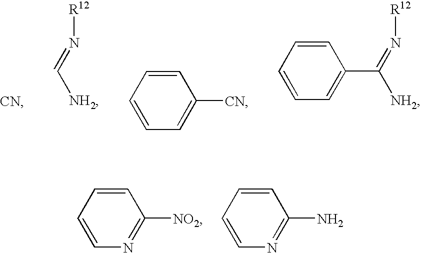
where R¹² is H, OH or O-alkyl;
R¹⁰ is H, CH₂—Ar—R⁹ where R⁹ is defined as above;
R¹ and R² taken together can form a compound of the formula IV 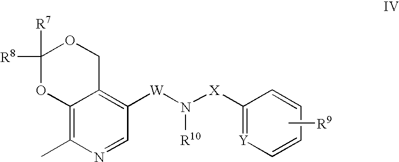
wherein W, X, Y, R⁷, R⁸, R⁹, and R¹⁰ are as described above; and
only one of R³ and R⁴ can be 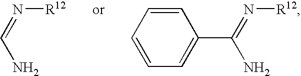
where R¹² is defined as above; or pharmaceutically acceptable salts thereof.

As used herein “alkyl” includes a saturated linear or branched hydrocarbon radical. In one embodiment, alkyl has from 1 to 8 carbon atoms. In another embodiment, alkyl has from 1 to 6 carbon atoms. In another embodiment, alkyl has from 1 to 4 carbon atoms. In one embodiment, alkyl has 1 carbon. The alkyl group may optionally be substituted with one or more substituents such as fluorine, chlorine, alkoxy groups having from 1 to 8 carbon atoms (e.g., methoxy or ethoxy), or amido groups having from 1 to 8 carbon atoms, such as acetamido. These substituents may themselves be substituted with one or more functional groups such as hydroxy groups, carboxy groups, acetoxy groups, or halogens.

As used herein “cycloalkyl” refers to a saturated hydrocarbon having from 3 to 8 carbon atoms, preferably 3 to 6 carbon atoms, such as, for example, cyclopropyl, cyclopentyl, cyclohexyl, and the like.

As used herein “aryl” means a mono- or poly-nuclear aromatic hydrocarbon radical. Examples of “aryl” groups include, but are not limited to aromatic hydrocarbons such as a phenyl group or a naphthyl group. The aromatic group may optionally be substituted with one or more substituents such as fluorine, chlorine, alkyl groups having from 1 to 8 carbon atoms (e.g., methyl or ethyl), alkoxy groups having from 1 to 8 carbon atoms (e.g., methoxy or ethoxy), alkoxyalkyl groups having from 1 to 8 carbon atoms and one or more oxygen atoms, or amido groups having from 1 to 8 carbon atoms, such as acetamido. These substituents may themselves be substituted with one or more functional groups such as hydroxy groups, carboxy groups, acetoxy groups, or halogens.

In one embodiment, aryl is a phenyl group or a naphthyl group that is either unsubstituted or substituted.

In another embodiment, aryl is a heteroaryl in which one or more of the carbon atoms of an aromatic hydrocarbon is substituted with a nitrogen, sulfur, or oxygen. Examples of a “heteroaryl” include, but are not limited to pyridine, pyrimidine, pyran, dioxin, oxazine, and oxathiazine. Likewise, the heteroaryl may optionally be substituted with functional groups such as hydroxy groups, carboxy groups, halogens, and amino groups.

As used herein, “amidine” means a group having the formula:

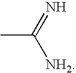

The invention also includes pharmaceutically acceptable salts of the compounds of the invention. The compounds of the invention are capable of forming both pharmaceutically acceptable acid addition and/or base salts. Pharmaceutically acceptable acid addition salts of the compounds of the invention include salts derived from nontoxic inorganic acids such as hydrochloric, nitric, phosphoric, sulfuric, hydrobromic, hydriodic, hydrofluoric, phosphorous, and the like, as well as the salts derived from nontoxic organic acids, such as aliphatic mono- and di-carboxylic acids, phenyl-substituted alkanoic acids, hydroxy alkanoic acids, alkanedioic acids, aromatic acids, aliphatic and aromatic sulfonic acids, etc. Such salts thus include sulfate, pyrosulfate, bisulfate, sulfite, bisulfite, nitrate, phosphate, monohydrogenphosphate, dihydrogenphosphate, metaphosphate, pyrophosphate, chloride, bromide, iodide, acetate, trifluoroacetate, propionate, caprylate, isobutyrate, oxalate, malonate, succinate, suberate, sebacate, fumarate, maleate, mandelate, benzoate, chlorobenzoate, methylbenzoate, dinitrobenzoate, phthalate, benzenesulfonate, toluenesulfonate, phenylacetate, citrate, lactate, maleate, tartrate, methanesulfonate, and the like. Also contemplated are salts of amino acids such as arginate and the like and gluconate, galacturonate, n-methyl glucamine, etc. (see Berge et al., J. Pharmaceutical Science, 66: 1-19 (1977). The term “pharmaceutically acceptable salts” also includes any pharmaceutically acceptable base salt including, but not limited to, amine salts, trialkyl amine salts and the like. Such salts can be formed quite readily by those skilled in the art using standard techniques.

The acid addition salts of the basic compounds are prepared by contacting the free base form with a sufficient amount of the desired acid to produce the salt in the conventional manner. The free base form may be regenerated by contacting the salt form with a base and isolating the free base in the conventional manner. The free base forms differ from their respective salt forms somewhat in certain physical properties such as solubility in polar solvents, but otherwise the salts are equivalent to their respective free base for purposes of the present invention. Base salts are formed with metals or amines, such as alkali and alkaline earth metals or organic amines. Examples of metals used as cations include, but are not limited to, sodium, potassium, magnesium, and calcium. Examples of suitable amines are N,N′-dibenzylethylenediamine, chloroprocaine, choline, diethanolamine, ethylenediamine, N-methylglucamine, and procaine.

Some of the compounds described herein contain one or more asymmetric centers and may thus give rise to enantiomers, diastereomers, and other stereoisomeric forms which may be defined in terms of absolute stereochemistry as (R)— or (S)—. The present invention is meant to include all such possible diastereomers and enantiomers as well as their racemic and optically pure forms. Optically active (R)- and (S)— isomers may be prepared using chiral synthons or chiral reagents, or resolved using conventional techniques. When the compounds described herein contain centers of geometric asymmetry, and unless specified otherwise, it is intended that the compounds include both E and Z geometric isomers. Likewise all tautomeric forms are intended to be included.

General Methods of Preparing Compounds of Formulae I, II, III, and IV

The compounds are generally prepared by combining an aldehyde or a carboxylate with an amine group to produce an elaborated pyridine structure. The general scheme of preparing the compounds of the formulae comprise protecting the hydroxyl groups at R₁ and R₂ of pyridoxine with known blocking groups such as esters, ethers, cyclic acetals, cyclic ketals, etc. and elaborating R³ through generating an aldehyde, acid, halide, or amine functionality as shown in schemes 1-4. R³ may be a nitro, amino, or cyano group that can be converted to an amidine by known chemical procedures. Additionally, protecting R¹ and R³ with known blocking groups such as esters, ethers, cyclic acetals, cyclic ketals, etc. and elaborating R² through generating an aldehyde, acid, halide, or amine functionality can be achieved through the same general scheme as shown in Scheme 5. 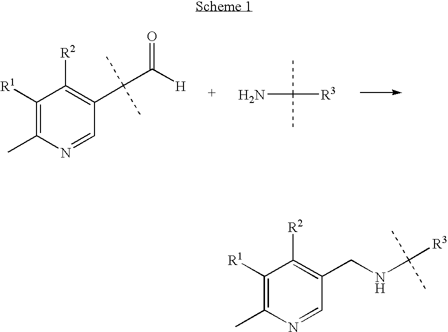
where the dashed lines are (CH₂)ₙ where n=0-8. 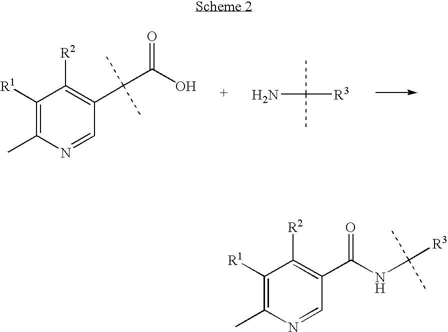
where the dashed lines are (CH₂)ₙ and n=0-8. 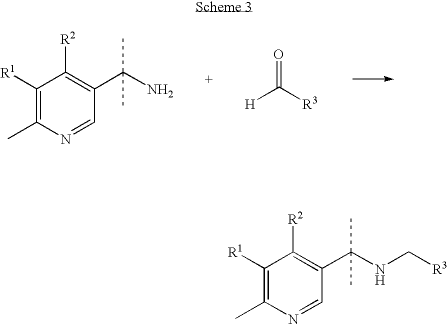
where the dashed lines are (CH₂)ₙ and n=0-8. 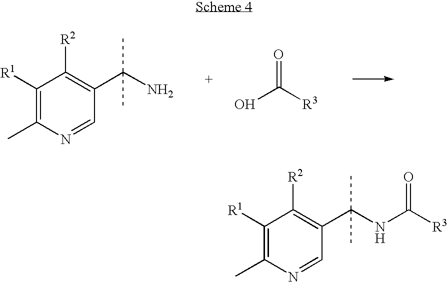
where the dashed lines are (CH₂)=and n=0-8. 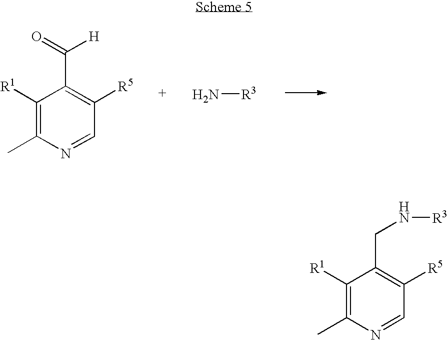
where R³ is (CH₂)ₙ—Ar—X, where n=0-8 and Ar—X is any aromatic terminating in a cyano, nitro, amidine, or amine.

Other positions on the pyridoxine ring can also be substituted according to the aforementioned general scheme. Substitutions are not specific to the positions described above.

Conditions to be Treated

In one embodiment of the invention, compounds of the invention can be used to treat cardiovascular or related diseases. Cardiovascular or related diseases include, for example, cerebral ischemia, cerebral hemorrhage, ischemic stroke, hemorrhagic stroke, hypertension, myocardial infarction, ischemia reperfusion injury, myocardial ischemia, congestive heart failure, blood coagulation disorders, cardiac hypertrophy, and platelet aggregation. Cardiovascular or related diseases also include diseases that arise from thrombotic and prothrombotic states in which the coagulation cascade is activated such as, for example, deep vein thrombosis, disseminated intravascular coagulopathy, and pulmonary embolism.

Heart failure is a pathophysiological condition in which the heart is unable to pump blood at a rate commensurate with the requirement of the metabolizing tissues or can do so only from an elevated filling pressure (increased load). Thus, the heart has a diminished ability to keep up with its workload. Over time, this condition leads to excess fluid accumulation, such as peripheral edema, and is referred to as congestive heart failure.

When an excessive pressure or volume load is imposed on a ventricle, myocardial hypertrophy (i.e., enlargement of the heart muscle) develops as a compensatory mechanism. Hypertrophy permits the ventricle to sustain an increased load because the heart muscle can contract with greater force. However, a ventricle subjected to an abnormally elevated load for a prolonged period eventually fails to sustain an increased load despite the presence of ventricular hypertrophy, and pump failure can ultimately occur.

Heart failure can arise from any disease that affects the heart and interferes with circulation. For example, a disease that increases the heart muscle's workload, such as hypertension, will eventually weaken the force of the heart's contraction. Hypertension is a condition in which there is an increase in resistance to blood flow through the vascular system. This resistance leads to increases in systolic pressure, diastolic blood pressure, or both. Hypertension places increased tension on the left ventricular myocardium, causing it to stiffen and hypertrophy, and accelerates the development of atherosclerosis in the coronary arteries. The combination of increased demand and lessened supply increases the likelihood of myocardial ischemia leading to myocardial infarction, sudden death, arrhythmias, and congestive heart failure.

Ischemia is a condition in which an organ or a part of the body fails to receive a sufficient blood supply. When an organ is deprived of a blood supply, it is said to be hypoxic. An organ will become hypoxic even when the blood supply temporarily ceases, such as during a surgical procedure or during temporary artery blockage. Ischemia initially leads to a decrease in or loss of contractile activity. When the organ effected is the heart, this condition is known as myocardial ischemia, and myocardial ischemia initially leads to abnormal electrical activity. This can generate an arrhythmia. When myocardial ischemia is of sufficient severity and duration, cell injury can progress to cell death—i.e., myocardial infarction—and subsequently to heart failure, hypertrophy, or congestive heart failure.

Ischemic reperfusion of the organ occurs when blood flow resumes to an organ after temporary cessation. For example, reperfusion of an ischemic myocardium can counter the effects of coronary occlusion, a condition that leads to myocardial ischemia. Ischemic reperfusion to the myocardium can lead to reperfusion arrhythmia or reperfusion injury. The severity of reperfusion injury is affected by numerous factors, such as, for example, duration of ischemia, severity of ischemia, and speed of reperfusion. Conditions observed with ischemia reperfusion injury include neutrophil infiltration, necrosis, and apoptosis.

Pharmaceutical Compositions

Although it is possible for compounds of the invention to be administered alone in a unit dosage form, the compounds are typically administered in admixture with a carrier as a pharmaceutical composition to provide a unit dosage form. The invention provides pharmaceutical compositions containing at least one compound of the invention. A pharmaceutical composition comprises a pharmaceutically acceptable carrier in combination with a compound of the invention or a pharmaceutically acceptable salt of a compound of the invention.

A pharmaceutically acceptable carrier includes, but is not limited to, physiological saline, ringers, phosphate-buffered saline, and other carriers known in the art. Pharmaceutical compositions can also include additives such as, for example, stabilizers, antioxidants, colorants, excipients, binders, thickeners, dispersing agents, readsorpotion enhancers, buffers, surfactants, preservatives, emulsifiers, isotonizing agents, and diluents. Pharmaceutically acceptable carriers and additives are chosen such that side effects from the pharmaceutical compound are minimized and the performance of the compound is not canceled or inhibited to such an extent that treatment is ineffective.

Methods of preparing pharmaceutical compositions containing a pharmaceutically acceptable carrier in combination with a therapeutic compound of the invention or a pharmaceutically acceptable acid addition salt of a compound of the invention are known to those of skill in the art. All methods can include the step of bringing the compound of the invention in association with the carrier and additives. The formulations generally are prepared by uniformly and intimately bringing the compound of the invention into association with a liquid carrier or a finely divided solid carrier or both, and then, if necessary, shaping the product into the desired unit dosage forms.

For oral administration as a tablet or capsule, the compositions can be prepared according to techniques well known in the art of pharmaceutical formulation. The compositions can contain microcrystalline cellulose for imparting bulk, alginic acid or sodium alginate as a suspending agent, methylcellulose as a viscosity enhancer, and sweeteners or flavoring agents. As immediate release tablets, the compositions can contain microcrystalline cellulose, starch, magnesium stearate and lactose or other excipients, binders, extenders, disintegrants, diluents and lubricants known in the art.

For administration by inhalation or aerosol, the compositions can be prepared according to techniques well known in the art of pharmaceutical formulation. The compositions can be prepared as solutions in saline, using benzyl alcohol or other suitable preservatives, absorption promoters to enhance bioavailability, fluorocarbons or other solubilizing or dispersing agents known in the art.

For administration as injectable solutions or suspensions, the compositions can be formulated according to techniques well-known in the art, using suitable dispersing or wetting and suspending agents, such as sterile oils, including synthetic mono- or di-glycerides, and fatty acids, including oleic acid.

For rectal administration as suppositories, the compositions can be prepared by mixing with a suitable non-irritating excipient, such as cocoa butter, synthetic glyceride esters or polyethylene glycols, which are solid at ambient temperatures, but liquefy or dissolve in the rectal cavity to release the drug.

Method of Treatment Using Compounds of the Invention

In another aspect of the invention, methods are provided for the treatment of cardiovascular or related diseases and symptoms thereof.

As used herein, the terms “treatment” and “treating” include inhibiting, alleviating, and healing cardiovascular or related diseases or symptoms thereof. Treatment can be carried out by administering a therapeutically effective amount of at least one compound of the invention. A “therapeutically effective amount” as used herein includes a prophylactic amount, for example an amount effective for alleviating or healing the above mentioned diseases or symptoms thereof.

A physician or veterinarian of ordinary skill readily determines a mammalian subject who is exhibiting symptoms of any one or more of the diseases described above. Regardless of the route of administration selected, a compound of the invention or a pharmaceutically acceptable acid addition salt of a compound of the invention can be formulated into pharmaceutically acceptable unit dosage forms by conventional methods known in the pharmaceutical art. An effective but nontoxic quantity of the compound is employed in treatment. The compounds can be administered in enteral unit dosage forms, such as, for example, tablets, sustained-release tablets, enteric coated tablets, capsules, sustained-release capsules, enteric coated capsules, pills, powders, granules, solutions, and the like. They can also be administered parenterally, such as, for example, subcutaneously, intramuscularly, intradermally, intramammarally, intravenously, and by other administrative methods known in the art.

The ordinarily skilled physician or veterinarian will readily determine and prescribe the therapeutically effective amount of the compound to treat the disease for which treatment is administered. In so proceeding, the physician or veterinarian could employ relatively low dosages at first, subsequently increasing the dose until a maximum response is obtained. Typically, the particular disease, the severity of the disease, the compound to be administered, the route of administration, and the characteristics of the mammal to be treated, for example, age, sex, and weight, are considered in determining the effective amount to administer. Administering a therapeutic amount of a compound of the invention for treating cardiovascular or related diseases or symptoms thereof, is in a range of about 0.1-100 mg/kg of a patient's body weight, more preferably in the range of about 0.5-50 mg/kg of a patient's body weight, per daily dose. The compound can be administered for periods of short and long duration. Although some individual situations can warrant to the contrary, short-term administration, for example, 30 days or less, of doses larger than 25 mg/kg of a patient's body weight is preferred to long-term administration. When long-term administration, for example, months or years, is required, the suggested dose usually does not exceed 25 mg/kg of a patient's body weight.

A therapeutically effective amount of a compound of the invention or a pharmaceutically acceptable addition salt of a compound of the invention for treating the above-identified diseases or symptoms thereof can be administered prior to, concurrently with, or after the onset of the disease or symptom. A compound of the invention can be administered concurrently. “Concurrent administration” and “concurrently administering” as used herein includes administering a compound of the invention and another therapeutic agent in admixture, such as, for example, in a pharmaceutical composition or in solution, or separately, such as, for example, separate pharmaceutical compositions or solutions administered consecutively, simultaneously, or at different times but not so distant in time such that the compound of the invention and the other therapeutic agent cannot interact and a lower dosage amount of the active ingredient cannot be administered.

In one embodiment of the invention, a method is provided for treating cardiovascular or related diseases comprising administering to a mammal a therapeutically effective amount of a compound of the invention or a pharmaceutically acceptable addition salt of a compound of the invention in a unit dosage form. The cardiovascular or related diseases that can be treated include hypertrophy, hypertension, congestive heart failure, heart failure subsequent to myocardial infarction, myocardial ischemia, cerebral ischemia, ischemia reperfusion injury, arrhythmia, myocardial infarction, blood coagulation, or platelet aggregation. Preferably, the cardiovascular disease treated is hypertrophy, congestive heart failure, arrhythmia, or ischemia reperfusion injury.

The compound of the invention can also be administered to treat cardiovascular diseases and other diseases that arise from thrombotic and prothrombotic states in which the coagulation cascade is activated, such as, for example, deep vein thrombosis, disseminated intravascular coagulopathy, Kasabach-Merritt syndrome, pulmonary embolism, myocardial infarction, stroke, thromboembolic complications of surgery, and peripheral arterial occlusion. A compound of the invention may also be useful in the treatment of adult respiratory distress syndrome, septic shock, septicemia, or inflammatory responses, such as edema and acute or chronic atherosclerosis, because thrombin has been shown to activate a large number of cells outside of the coagulation process, such as, for example, neutrophils, fibroblasts, endothelial cells, and smooth muscle cells.

The method for treating cardiovascular or related diseases can further comprise concurrent administration of other therapeutic agents already known to be suitable for treating the above-identified diseases. For example, methods of the invention include concurrently administering a compound of the invention or a pharmaceutically acceptable acid addition salt of a compound of the invention in combination with a therapeutic cardiovascular compound to treat hypertrophy, hypertension, congestive heart failure, heart failure subsequent to myocardial infarction, myocardial ischemia, ischemia reperfusion injury, arrhythmia, or myocardial infarction. Preferably, the cardiovascular disease treated is hypertrophy, congestive heart failure, arrhythmia, or ischemia reperfusion injury.

The compounds of the invention can also be used in combination with other therapeutic cardiovascular compounds that are generally used to treat cardiovascular or related diseases as well as symptoms thereof. A skilled physician or veterinarian readily determines a subject who is exhibiting symptoms of any one or more of the diseases described above and makes the determination about which compound is generally suitable for treating specific cardiovascular conditions and symptoms.

For example, myocardial ischemia can be treated by the administration of a compound of the invention or a pharmaceutically acceptable acid addition salt of a compound of the invention concurrently with another therapeutic agent. Other suitable therapeutic agents include, for example, a angiotensin converting enzyme inhibitor, an angiotensin II receptor antagonist, a calcium channel blocker, an antithrombolytic agent, a β-adrenergic receptor antagonist, a diuretic, an α-adrenergic receptor antagonist, or a mixture thereof.

As another example, congestive heart failure can be treated by the administration of a compound of the invention or a pharmaceutically acceptable acid addition salt of a compound of the invention concurrently with another therapeutic agent. Other suitable therapeutic agents include, for example, an angiotensin converting enzyme inhibitor, an angiotensin II receptor antagonist, a calcium channel blocker, a vasodilator, a diuretic, or a mixture thereof.

Myocardial infarction can be treated by the administration of a compound of the invention or a pharmaceutically acceptable acid addition salt of a compound of the invention concurrently with another therapeutic agent. Other suitable therapeutic agents include, for example, a angiotensin converting enzyme inhibitor, a calcium channel blocker, an antithrombolytic agent, a β-adrenergic receptor antagonist, a diuretic, an α-adrenergic receptor antagonist, or a mixture thereof.

Hypertension can be treated by the administration of a compound of the invention or a pharmaceutically acceptable acid addition salt of a compound of the invention concurrently with another therapeutic agent. Other suitable therapeutic agents include, for example, an angiotensin converting enzyme inhibitor, a calcium channel blocker, a β-adrenergic receptor antagonist, a vasodilator, a diuretic, an α-adrenergic receptor antagonist, or a mixture thereof.

Arrhythmia can be treated by the administration of a compound of the invention or a pharmaceutically acceptable acid addition salt of a compound of the invention concurrently with another therapeutic agent. Other suitable therapeutic agents include, for example, a calcium channel blocker, an β-adrenergic receptor antagonist, or a mixture thereof.

Blood clots in the arteries (arterial thrombosis) or veins (venous thrombosis) can be reduced or removed by the administration of a compound of the invention or a pharmaceutically acceptable acid addition salt of a compound of the invention concurrently with a anti-platelet agent such as clopidogrel, aspirin, dipyridamole, etc., glycoprotein IIb/IIIa inhibitor such as integrillin etc., or by anticoagulant such as UFH (unfractionated heparins) or LMWH (low molecular weight heparins) or by hirudin or argatroban etc.

Hypertrophy can be treated by the administration of a compound of the invention or a pharmaceutically acceptable acid addition salt of a compound of the invention concurrently with another therapeutic agent. Other suitable therapeutic agents include, for example, an angiotensin converting enzyme inhibitor, an angiotensin II receptor antagonist, a calcium channel blocker, or a mixture thereof.

Ischemia reperfusion injury can be treated by the administration of a compound of the invention or a pharmaceutically acceptable acid addition salt of a compound of the invention concurrently with another therapeutic agent. Other suitable therapeutic agents include, for example, an angiotensin converting enzyme inhibitor, an angiotensin II receptor antagonist, a calcium channel blocker, or a mixture thereof.

Compounds of the invention or pharmaceutically acceptable salts thereof can be administered post-surgically, alone or concurrently with other suitable therapeutic agents. For instance, the method would include, but is not limited to, administration to patients following hip replacement surgery, or invasive cardiovascular surgery, including coronary artery bypass graft (CABG), endarectomy, and heart valve replacement. Compounds of the invention or pharmaceutically acceptable salts thereof can be administered, alone or concurrently with other suitable therapeutic agents, following any angioplasty procedure. For instance, administration of said compounds may follow percutaneous transluminal angioplasty (PTA). PTA is used in coronary, pulmonary, peripheral, intracranial, extracranial carotid, renal, and aortic stenoses.

Additionally, medical devices can be coated with the compounds of the invention or pharmaceutically acceptable acid salts of the compound alone or in mixture with other suitable therapeutic agents (e.g., an angiotensin converting enzyme inhibitor). Medical devices that can be coated with the compounds of the invention or pharmaceutically acceptable salts thereof alone or in mixture with other suitable therapeutic agents include, but are not limited to, intravascular stents and catheters. Intravascular stents are used to prevent blood vessel wall collapse. Drug-eluting stents are coated with a mixture of polymers and drug to prevent restenosis. Examples of drug-eluting stents are the CYPHER™ sirolimus-eluting stent (Cordis Corp., Miami, Fla.) and TAXUS™ paclitaxel-eluting stent (Boston Scientific Corp., Natick, Mass.).

This invention is further characterized by the following examples. These examples are not meant to limit the scope of the invention but are provided for exemplary purposes to more fully describe the invention. Variation within the scope of the invention will be apparent to those skilled in the art.

### EXAMPLES

All reagents used were purchased from standard commercial sources, or synthesized by known literature methods. HPLC analysis was performed using a Water 996 PDA High performance Liquid chromatograph equipped with a Water 600 controller. Signals were detected with a photodiode array detector (set at max plot 254-400 nm). NMR spectra were recorded on a Bruker AM-300 instrument (¹³C, ¹⁹F and ³¹P at 75.5, 282 and 121 MHz respectively) and were calibrated using residual nondeuterated solvent as the internal reference. All ¹⁹F spectra are reported using hexafluorobenzene (δ-162.9 ppm) as the external standard while ³¹P spectra were collected using 85% H₃PO₄ (δ0.0 ppm) as the external reference.

### Example 1

### Synthesis of 3-Cyano-N-(2,2,8-trimethyl-4H-[1,3]dioxino[4,5-c]pyridine-5-ylmethyl)-benzamide (1)

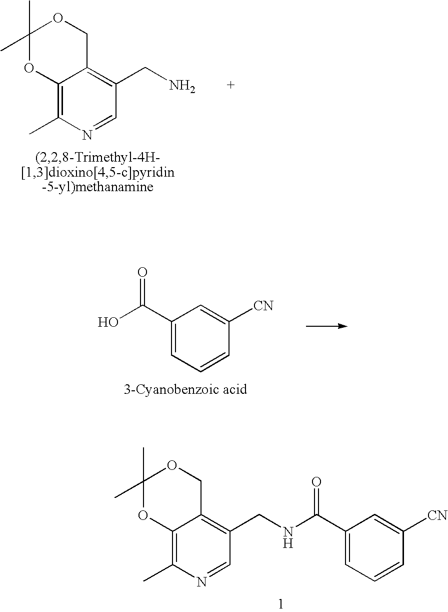

A mixture of (2,2,8-trimethyl-4H-[1,3]dioxino[4,5-c]pyridin-5-yl)methanamine (1.00 g, 4.80 mmol), 3-cyanobenzoic acid (853 mg, 5.80 mmol), 1-[3-(dimethylamino)propyl]-3-ethylcarbodiimide hydrochloride (EDC) (1.38 g, 7.20 mmol), and N,N-dimethylaminopyridine (DMAP) (586 mg, 4.80 mmol) in anhydrous N,N-dimethylformamide (DMF, 100 mL) was stirred at room temperature overnight. The reaction mixture was then extracted with diethyl ether (5×100 mL) and the ethereal layer was washed several times with water. The combined organic layer was dried over anhydrous magnesium sulfate, filtered and evaporated to give a crude mixture, then purified by column chromatography on silica gel to give 3-cyano-N-(2,2,8-trimethyl-4H-[1,3]dioxino[4,5-c]pyridine-5-ylmethyl)-benzamide (1) (800 mg, 49% yield) as a colorless solid.

¹H-NMR (CDCl₃): δ 8.09-8.05 (m, 1H), 8.07-8.01 (m, 2H), 7.81-7.78 (m, 1H), 7.60-7.55 (m, 1H), 6.45-6.30 (m, 1H), 4.89 (s, 2H), 4.53 (d, 2H), 2.40 (s, 3H), 1.55 (s, 6H).

### Example 2

### Synthesis of 3-Carbamimidoyl-N-(5-hydroxy-4-hydroxymethyl-6-methyl-pyridin-3-ylmethyl)-benzamide (2)

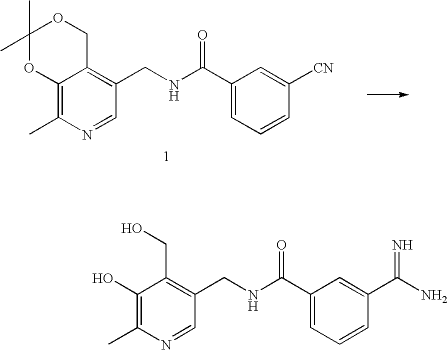

Hydrogen chloride gas was bubbled into a suspension of 3-cyano-N-(2,2,8-trimethyl-4H-[1,3]dioxino[4,5-c]pyridine-5-ylmethyl)-benzamide (1) (600 mg, 1.78 mmol) in absolute ethyl alcohol (100 mL) at room temperature for 45 minutes. The solid dissolved instantly and the mixture turned to a clear yellow solution. The septum was replaced and the reaction mixture was stirred at room temperature overnight. The remaining hydrogen chloride gas was removed by purging with nitrogen gas for 2 hours, and the solvent evaporated to give the crude amide ester as a yellow solid. Ammonia in methyl alcohol (50 mL, 7 M, 350 mmol) was added to the crude amide ester and stirred overnight at room temperature. The solvent was evaporated and the product purified on a silica gel column using a mixture of isopropanol:water:30% ammonium hydroxide (4:1:1) as eluant to give the corresponding benzamide 3-carbamimidoyl-N-(5-hydroxy-4-hydroxymethyl-6-methyl-pyridin-3-ylmethyl)-benzamide (2) (139 mg, 25% yield) as a light yellow solid.

¹H-NMR (CD₃OD): δ 8.28-8.25 (m, 1H), 8.18-8.13 (m, 1H), 7.96-7.91 (m, 1H), 7.87-7.83 (m, 1H), 7.73-7.68 (m, 1H), 4.96 (m, 2H), 4.61 (m, 2H), 2.40 (s, 3H).

### Example 3

### Synthesis of 4-Cyano-N-(2,2,8-trimethyl-4H-[1,3]dioxino[4,5-c]pyridine-5-ylmethyl)-benzamide (3)

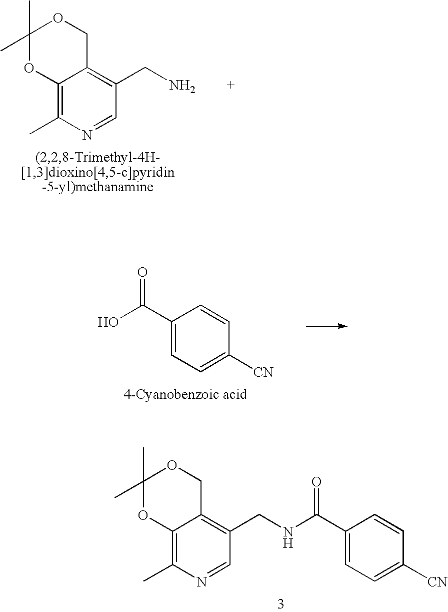

The coupling of (2,2,8-trimethyl-4H-[1,3]dioxino[4,5-c]pyridin-5-yl)methanamine (1.00 g, 4.80 mmol) and 4-cyanobenzoic acid (706 mg, 4.80 mmol), as described in Example 1, gave a colorless solid 4-cyano-N-(2,2,8-trimethyl-4H-[1,3]dioxino[4,5-c]pyridine-5-ylmethyl)-benzamide (3) (1.57 g, 95% yield).

¹H-NMR (CDCl₃): δ 7.93 (s, 1H), 7.91-7.86 (m, 2H), 7.76-7.70 (m, 2H), 4.87 (s, 2H), 4.51 (d, 2H), 2.37 (s, 3H), 1.54 (s, 6H).

### Example 4

### Synthesis of 4-Cyano-N-(5-hydroxy-4-hydroxymethyl-6-methyl-pyridin-3-ylmethyl)-benzamide (4)

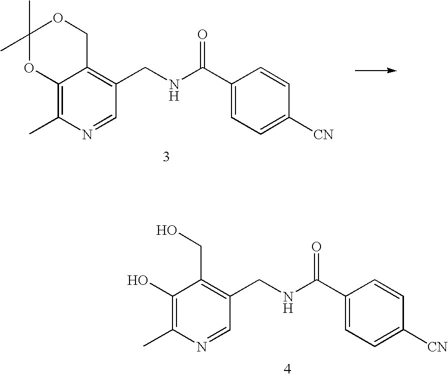

4-Cyano-N-(2,2,8-trimethyl-4H-[1,3]dioxino[4,5-c]pyridine-5-ylmethyl)-benzamide (3) was heated in a solution of 10% formic acid in water for 2 hours at 100° C. After evaporating the solvent, the crude product was washed with dichloromethane to give 4-cyano-N-(5-hydroxy-4-hydroxymethyl-6-methyl-pyridin-3-ylmethyl)-benzamide (4) (87 mg, 99% yield).

¹H-NMR (CD₃OD): δ 7.99-7.93 (m, 2H), 7.93-7.90 (m, 1H), 7.86-80 (m, 2H), 4.97 (s, 2H), 4.60 (s, 2H), 2.43 (s, 3H).

### Example 5

### Synthesis of 4-Carbamimidoyl-N-(5-hydroxyl-4-hydroxymethyl-6-methyl-pyridin-3-ylmethyl)-benzamide (5)

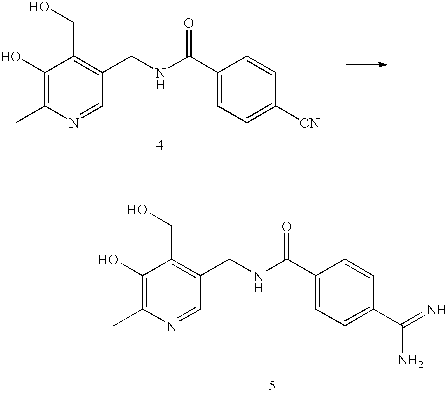

The conversion of nitrile (4) to amidine (5) was carried out as described in Example 2.

¹H-NMR (DMSO-d₆): δ 8.09 (m, 1H), 8.06 (m, 2H), 8.04 (m, 2H), 4.94 (s, 1H), 4.66-4.64 (s, 2H), 2.50 (s, 3H).

### Example 6

### Synthesis of 4-{[2,2,8-Trimethyl-4H-[1,3]dioxino[4,5-c]pyridin-5-ylmethyl)-amino]-methyl}-benzonitrile (6)

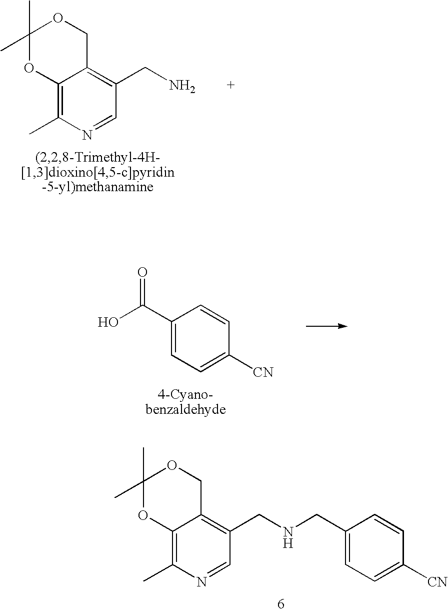

In a 250 mL three-necked round bottom flask fitted with a condenser and Dean-Stark trap, a mixture of (2,2,8-trimethyl-4H-[1,3]dioxino[4,5-c]pyridin-5-yl)methanamine (300 mg, 1.44 mmol), 4-cyanobenzaldehyde (189 mg, 1.44 mmol), and a catalytic amount of p-toluenesulfonic acid (27 mg, 0.14 mmol) in benzene (15 mL) was heated at 100° C. under nitrogen atmosphere for 18 hours. The solvent mixture was then evaporated and the crude product was dissolved in methanol (20 mL), followed by the addition of sodium borohydride (163 mg, 4.32 mmol). The reaction mixture was stirred at room temperature for 2 hours, quenched with saturated aqueous sodium bicarbonate (40 mL), extracted with ethyl acetate (2×100 mL) and then back washed with water (2×00 mL). The combined organic layer was dried over anhydrous magnesium sulfate, filtered and evaporated. The crude product was purified by column chromatography on silica gel using dichloromethane:methyl alcohol (5:1) as eluant to give the compound 4-{[2,2,8-trimethyl-4H-[1,3]dioxino[4,5-c]pyridin-5-ylmethyl)-amino]-methyl}-benzonitrile (6) as a colorless solid (265 mg, 57% overall yield for two steps).

¹H-NMR (CDCl₃): δ 7.92 (m, 1H), 7.63-7.62 (m, 2H), 7.60 (m, 2H), 4.93 (s, 2H), 3.85 (s, 2H), 3.66 (s, 2H), 2.39 (s, 3H), 1.57 (s, 6H).

### Example 7

### Synthesis of 4-{[(5-Hydroxyl-4-hydroxymethyl-6-methyl-pyridin-3-ylmethyl)-amino]-methyl}-benzamidine (7)

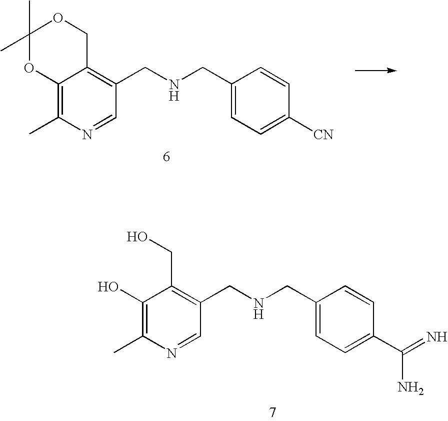

The conversion of nitrile (6) to amidine (7) was carried out as described in Example 2.

¹H-NMR (CD₃OD): δ 7.94 (m, 1H), 7.86-7.83 (m, 2H), 7.71-7.69 (m, 2H), 4.18 (s, 2H), 4.11 (s, 2H), 2.45 (s, 3H).

### Example 8

### Synthesis of 3-{1[(2,2,8-Trimethyl-4H-[1,3]dioxino[4,5-c]pyridin-5-ylmethyl)-amino]-methyl}-benzonitrile (8)

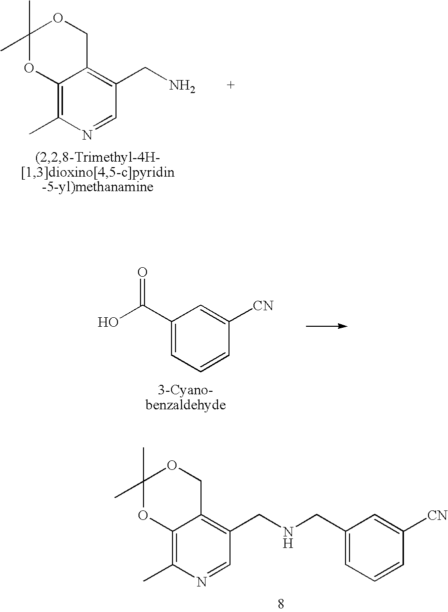

The reductive amination of (2,2,8-trimethyl-4H-[1,3]dioxino[4,5-c]pyridin-5-yl)methanamine (1.0 g, 4.80 mmol) and 3-cyanobenzaldehyde (630 mg, 4.80 mmol), as described in Example 6, gave a yellow solid 3-{[(2,2,8-trimethyl-4H-[1,3]dioxino[4,5-c]pyridin-5-ylmethyl)-amino]-methyl}-benzonitrile (8) (621 mg, 40% yield).

¹H-NMR (CDCl₃): δ 7.92 (m, 1H), 7.61 (m, 1H), 7.57-7.55 (m, 2H), 7.54-7.53 (m, 1H), 4.92 (s, 2H), 3.82 (s, 2H), 3.66 (s, 2H), 2.39 (s, 3H), 1.56 (m, 7H).

### Example 9

### Synthesis of 3-{[(5-Hydroxy-4-hydroxymethyl-6-methyl-pyridin-3-ylmethyl)-amino]-methyl]-benzamidine (9)

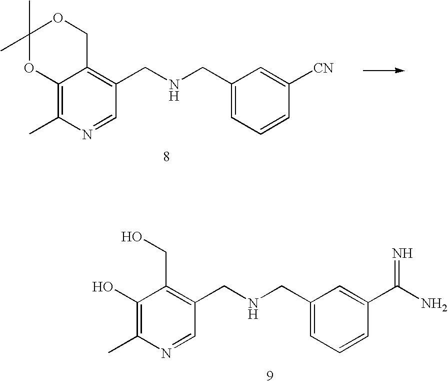

The conversion of nitrile (8) to amidine (9) was carried out as described in Example 2.

¹H-NMR (CD₃OD): δ 8.01 (m, 1H), 7.97-7.96 (m, 1H), 7.88-7.81 (m, 2H), 7.72-7.67 (m, 1H), 4.95 (m, 2H), 4.30 (s, 2H), 4.25 (s, 2H), 2.46 (s, 3H).

### Example 10

### Synthesis of N-(3-Cyanobenzyl)-2,2,8-trimethyl-4H-[1,3]dioxino[4,5-c]pyridine-5-carboxamide

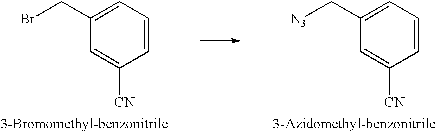

Step 1: A mixture of 3-bromomethyl-benzonitrile (20.0 g, 0.102 mol) and sodium azide (66.3 g, 1.02 mol) in anhydrous DMF (200 mL) was stirred at room temperature overnight. Water (100 mL) was added to the reaction mixture, and the mixture was then extracted with diethyl ether (3×100 mL). The combined organic layer was dried over anhydrous magnesium sulfate, filtered and evaporated to give 3-azidomethyl-benzonitrile as a colorless solid (12.4 g, 77% yield).

¹H-NMR (CD₃OD): δ 7.77-7.66 (m, 3H), 7.63-7.55 (m, 1H), 4.82 (s, 2H).

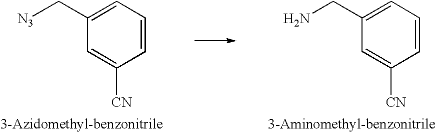

Step 2: The 3-azidomethyl-benzonitrile (12.4 g, 0.078 mol) in ethyl acetate (40 mL) was hydrogenated at 45 psi in the presence of 5% palladium on carbon (4.0 g) at room temperature overnight. The product was filtered through a celite pad and the solvent was evaporated to give 3-aminomethyl-benzonitrile as light brown solid (7.87 g, 76% yield).

¹H-NMR (CDCl₃): δ 7.62-7.57 (m, 1H), 7.56-7.43 (m, 2H), 7.42-7.31 (m, 1H), 3.87 (s, 2H).

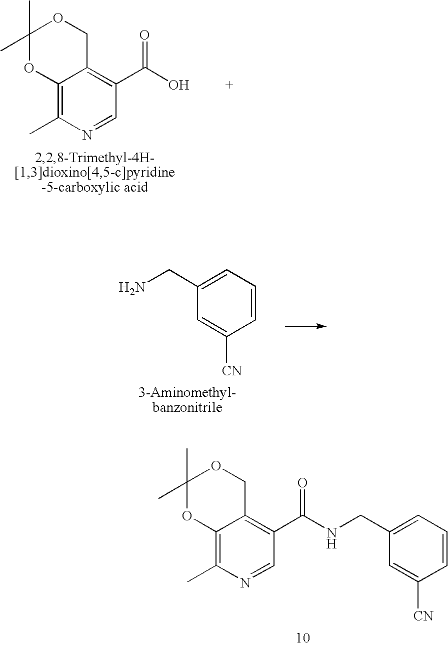

Step 3: The coupling of 2,2,8-trimethyl-4H-[1,3]dioxino[4,5-c]pyridine-5-carboxylic acid (1.69 g, 7.60 mmol) and 3-aminomethyl-benzonitrile (1.00 g, 7.60 mmol), as described in Example 1, gave colorless solid N-(3-cyanobenzyl)-2,2,8-trimethyl-4H-[1,3]dioxino[4,5-c]pyridine-5-carboxamide (10) (0.93 g, 36% yield).

¹H-NMR (CDCl₃): δ 8.22 (m, 1H), 7.60 (m, 3H), 7.47 (m, 1H), 5.09 (s, 2H), 4.63 (s, 2H), 2.43 (s, 3H), 1.56 (m, 6H).

### Example 11

### Synthesis of N-(3-Cyano-benzyl)-5-hydroxy-4-hydroxymethyl-6-methyl-nicotinamide (11)

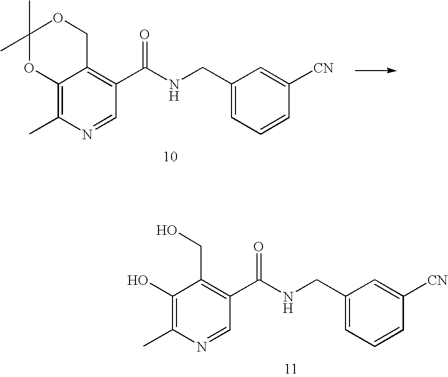

The hydrolysis of N-(3-cyanobenzyl)-2,2,8-trimethyl-4H-[1,3]dioxino[4,5-c]pyridine-5-carboxamide (10) (900 mg, 2.67 mmol), as described in Example 4, gave N-(3-cyano-benzyl)-5-hydroxy-4-hydroxymethyl-6-methyl-nicotinamide (11) (769 mg, 97% yield) as a light yellow solid.

### Example 12

### Synthesis of 5-Hydroxy-N-[3-(N-hydroxycarbamimidoyl)-benzyl]-4-hydroxymethyl-6-methyl-nicotinamide (12)

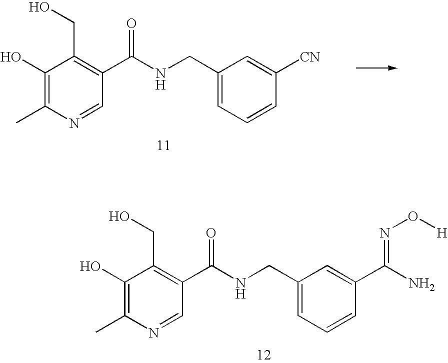

A mixture of N-(3-cyano-benzyl)-5-hydroxy-4-hydroxymethyl-6-methyl-nicotinamide (11) (200 mg, 0.67 mmol), hydroxylamine hydrochloride (90 mg, 1.35 mmol), and N,N-Diisopropyl-ethylamine (DIEA) (0.10 mL) was stirred in methyl alcohol at room temperature for 16 hours. The crude mixture was evaporated and purified by column chromatography on silica gel using a mixture of dichloromethane:methyl alcohol (10:1) as eluant to give 5-hydroxy-N-[3-(N-hydroxycarbamimidoyl)-benzyl]-4-hydroxymethyl-6-methyl-nicotinamide (12) (210 mg, 91% yield) as a colorless solid.

¹H-NMR (DMSO-d₆): δ 8.18 (m, 1H), 8.08-8.06 (m, 2H), 7.93-7.90 (m, 2H), 4.99 (s, 2H), 4.74-4.72 (m, 2H), 2.64 (s, 3H).

### Example 13

### Synthesis of 3-[(2,2,8-Trimethyl-4H-[1,3]dioxino[4,5-c]pyridin-5-ylmethyl)-amino]-benzonitrile (13)

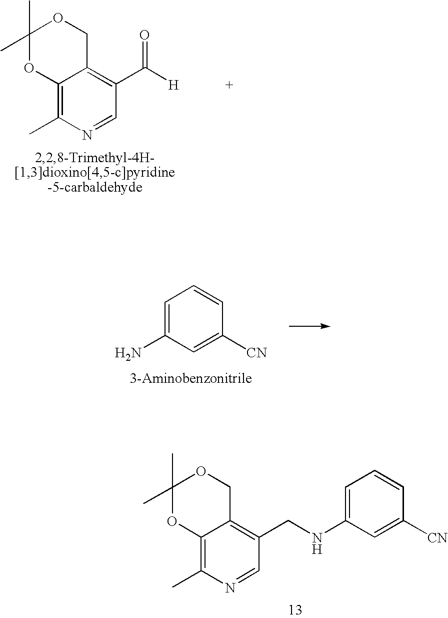

The reductive amination of 3-aminobenzonitrile (6.97 g, 59 mmol) and 2, 2,8-trimethyl-4H-[1,3]dioxino[4,5-c]pyridine-5-carbaldehyde (13.5 g, 65 mmol), as described in Example 6, gave 3-[(2,2,8-trimethyl-4H-[1,3]dioxino[4,5-c]pyridin-5-ylmethyl)-amino]-benzonitrile (13) (3.65 g, 20% yield).

¹H-NMR (CDCl₃): δ 8.00 (s, 1H), 7.24 (m, 1H), 7.01 (d, 1H), 6.84 (s, 1H), 6.82 (d, 1H), 4.86 (s, 1H), 4.16 (d, 2H), 4.09 (m, 1H), 2.42 (s, 3H), 1.56, (s, 6H).

### Example 14

### Synthesis of 3-[(5-Hydroxy-4-hydroxymethyl-6-methyl-pyridin-3-ylmethyl)-amino]-benzamidine (14)

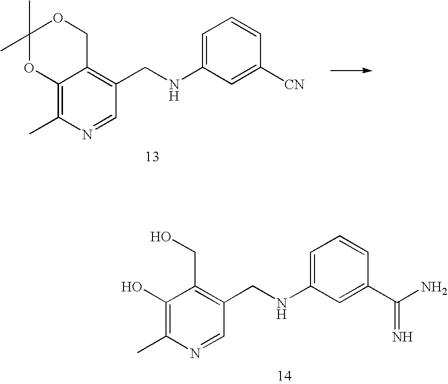

The conversion of nitrile (13) to amidine (14) was carried out as shown in Example 2.

¹H-NMR (CD₃OD): δ 7.86 (s, 1H), 7.33 (t, 1H), 6.98 (m, 3H), 4.96 (s, 2H), 4.38 (s, 2H), 2.42 (s, 3H). MS (ES⁺) m/z: 287.15 (M+H⁺).

### Example 15

### Synthesis of 4-(6-Hydroxyamino-pyridine-3-yl)-N-(2,2,8-trimethyl-4H-[1,3]dioxino[4,5-c]pyridine-5-ylmethyl)-benzamide (15)

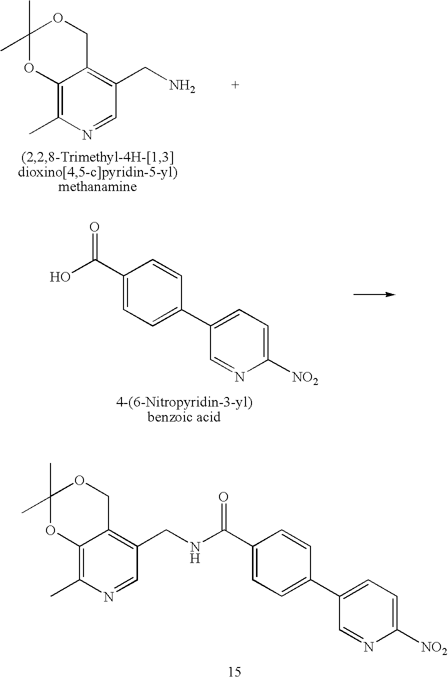

The coupling of (2,2,8-trimethyl-4H-[1,3]dioxino[4,5-c]pyridin-5-yl)methanamine (1.00 g, 4.80 mmol) and 4-(6-nitro-pyridin-3-yl)-benzoic acid (2.34 g, 9.60 mmol), performed as described in Example 1, gave 4-(6-hydroxyamino-pyridine-3-yl)-N-(2,2,8-trimethyl-4H-[1,3]dioxino[4,5-c]pyridine-5-ylmethyl)-benzamide (15) (1.62 g, 78% yield) as a yellow solid.

¹H-NMR (CDCl₃): δ 8.85 (m, 1H), 8.37 (m, 1H), 8.21 (m, 1H), 7.97 (m, 3H), 7.71 (m, 2H), 4.91 (s, 2H), 4.55 (s, 2H), 2.40 (s, 3H), 1.55 (s, 6H).

### Example 16

### Synthesis of 4-(6-Amino-pyridin-3-yl)-N-(2,2,8-trimethyl-4H-[1,3]dioxino[4,5-c]pyridin-5-ylmethyl)-benzamide (16)

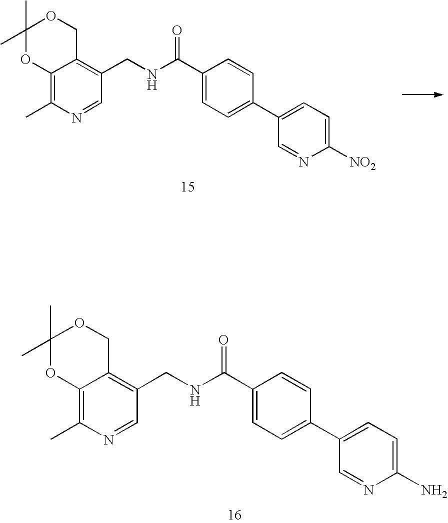

A mixture of 4-(6-hydroxyamino-pyridine-3-yl)-N-(2,2,8-trimethyl-4H-[1,3]dioxino[4,5-c]pyridine-5-ylmethyl)-benzamide (15) (200 mg, 0.46 mmol) in ethyl acetate (45 mL) was hydrogenated at room temperature with 10% palladium on carbon (800 mg) at a pressure of 20 psi. The product was then filtered through a celite pad and the solvent was evaporated to give the light yellow solid 4-(6-amino-pyridin-3-yl)-N-(2,2,8-trimethyl-4H-[1,3]dioxino[4,5-c]pyridin-5-ylmethyl)-benzamide (16) (66 mg, 36% yield).

¹H-NMR (CD₃OD): δ 8.25 (s, 1H), 7.98 (s, 1H), 7.91 (d, 2H), 7.83 (s, 1H), 7.82 (d, 2H), 6.72-6.69 (m, 1H), 5.02 (s, 2H), 4.50 (s, 2H), 2.37 (s, 3H), 1.57 (m, 6H).

### Example 17

### Synthesis of 4-(6-Amino-pyridin-3-yl)-N-(5-hydroxy-4-hydroxymethyl-6-methyl-pyridin-3-ylmethyl)-benzamide (17)

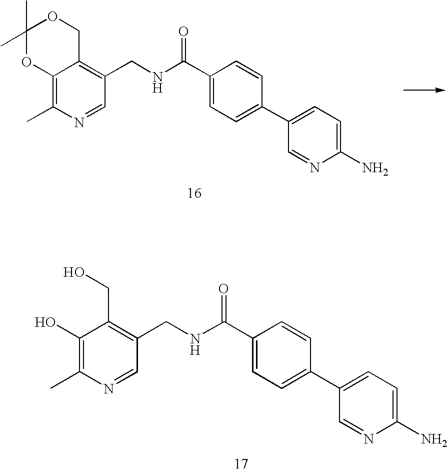

The hydrolysis of 4-(6-amino-pyridin-3-yl)-N-(2,2,8-trimethyl-4H-[1,3]dioxino[4,5-c]pyridin-5-ylmethyl)-benzamide (16) (66 mg, 0.16 mmol), as described in Example 4, gave 4-(6-amino-pyridin-3-yl)-N-(5-hydroxy-4-hydroxymethyl-6-methyl-pyridin-3-ylmethyl)-benzamide (17) (53 mg, 89% yield) as a colorless solid.

¹H-NMR (DMSO-d₆): δ 8.99 (m, 1H), 8.51-8.50 (m, 1H), 8.31 (m, 1H), 8.09 (m, 3H), 7.98-7.94 (m, 1H), 7.83 (m, 2H), 4.96 (s, 2H), 4.67 (s, 2H), 2.52 (s, 3H).

### Example 18

### Synthesis of N-(5-Hydroxy-4-hydroxymethyl-6-methyl-pyridin-3-ylmethyl)-4-(6-nitro-pyridin-3-yl)-benzamide (18)

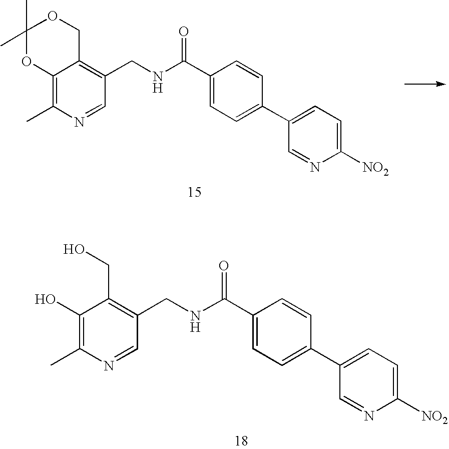

The hydrolysis of 4-(6-hydroxyamino-pyridine-3-yl)-N-(2,2,8-trimethyl-4H-[1,3]dioxino[4,5-c]pyridine-5-ylmethyl)-benzamide (15) (250 mg, 0.575 mmol), carried out as described in Example 4, gave N-(5-hydroxy-4-hydroxymethyl-6-methyl-pyridin-3-ylmethyl)-4-(6-nitro-pyridin-3-yl)-benzamide (18) (221 mg, 97% yield) as a colorless solid.

¹H-NMR (DMSO-d₆): δ 9.07 (m, 1H), 8.97 (m, 1H), 8.61-8.57 (m, 1H), 8.43-8.40 (m, 1H), 8.06-7.97 (m, 3H), 7.92 (m, 1H), 4.79 (s, 2H), 4.53 (s, 2H), 2.34 (s, 3H).

### Example 19

### Synthesis of 4′-Cyano-biphenyl-4-carboxylic acid (2,2,8-trimethyl-4H-[1,3]dioxino[4,5-c]pyridine-5-ylmethyl)-amide (19)

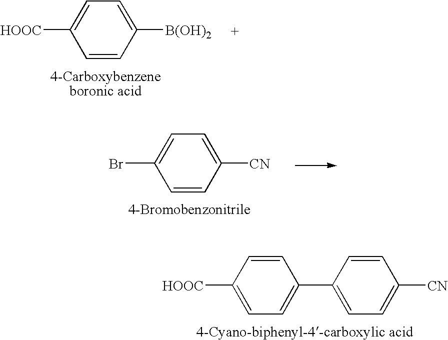

Step 1: A mixture of 4-carboxybenzeneboronic acid (4.0 g, 24 mmol), 4-bromobenzonitrile (4.40 g, 24.1 mmol), sodium carbonate (5.20 g, 48.2 mmol), and palladium on carbon (1.20 g) in 1:1 methanol:water mixture (100 mL) was heated at 77° C. overnight. The mixture was filtered through a celite pad and and the pad was washed with a mixture of 1:1 methanol:water (400 mL). The solvent was partly evaporated and adjusted to a pH of about 4.0-4.5 by adding dropwise 1N hydrochloric acid to precipitate the product. The product was collected by filtration, and washed with water to give 4′-cyano-biphenyl-4-carboxylic acid as a colorless solid (5.28 g, 98% yield).

¹H-NMR (DMSO-d₆): δ 8.05-8.03 (m, 2H), 7.95 (m, 4H), 7.86-7.84 (m, 2H).

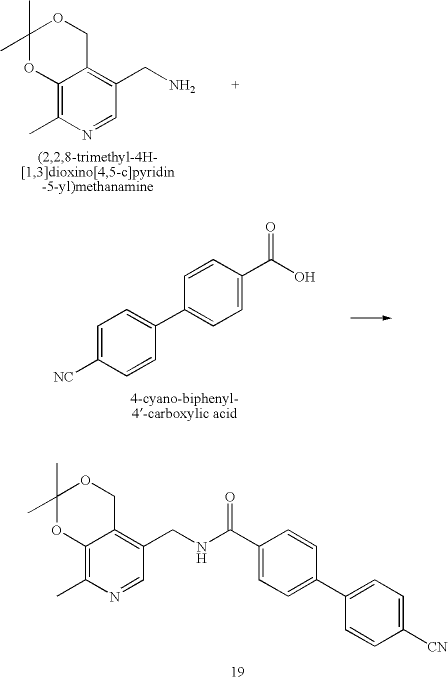

Step 2: A mixture of 4′-cyano-biphenyl-4-carboxylic acid (5.0 g, 22.40 mmol), (2,2,8-trimethyl-4H-[1,3]dioxino[4,5-c]pyridin-5-yl)methanamine (9.33 g, 44.80 mmol), EDC (8.60 g, 44.80 mmol), and 1-hydroxybenzotriazole hydrate (6.05 g, 44.80 mmol) in anhydrous DMF (100 mL) was stirred at room temperature overnight. Water (200 mL) was added and the crude product was extracted with diethyl ether (700 mL), the organic solution then back washed with water (500 mL). The combined organic layer was dried over anhydrous magnesium sulfate, filtered and evaporated to give a crude mixture which was purified by column chromatography on silica gel to give 4′-cyano-biphenyl-4-carboxylic acid (2,2,8-trimethyl-4H-[1,3]dioxino[4,5-c]pyridine-5-ylmethyl)-amide (19) (9.03 g, quantitative yield) as a light yellow solid.

¹H-NMR (CDCl₃): δ 8.14 (m, 1H), 7.95-7.93 (m, 2H), 7.80-7.55 (m, 6H), 6.99 (s br, 1H), 4.98 (s, 2H), 4.56 (s, 2H), 2.46 (s, 3H), 1.56 (s, 6H).

### Example 20

### Synthesis of 4′-Carbamimidoyl-biphenyl-4-carboxylic (5-hydroxyl-4-hydroxymethyl-6-methyl-pyridin-3-ylmethyl)-amide (20)

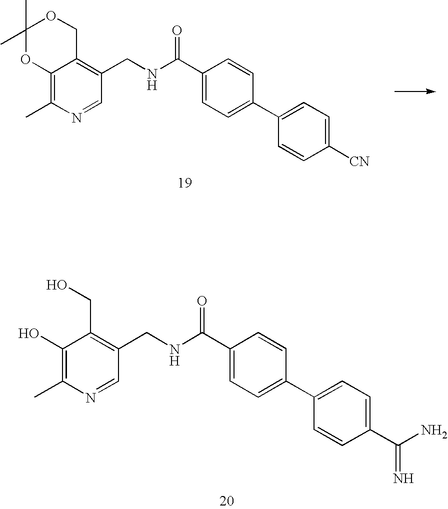

The conversion of nitrile (19) to amidine (20) was carried out as described in Example 2.

¹H-NMR (DMSO-d₆): δ 8.87 (m, 1H), 7.98-7.95 (m, 2H), 7.91-7.88 (m, 2H), 7.83-7.79 (m, 5H), 4.76 (s, 2H), 4.48-4.47 (s, 2H), 2.3 (s, 3H).

### Example 21

### Synthesis of 4′-Cyano-biphenyl-4-carboxylic acid (5-hydroxy-4-hydroxymethyl-6-methyl-pyridin-3-ylmethyl)-amide (21)

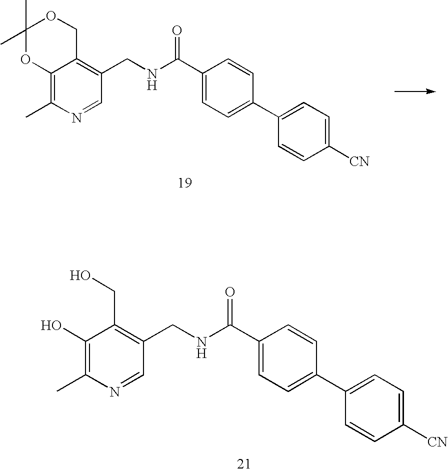

The hydrolysis of 4′-cyano-biphenyl-4-carboxylic acid (2,2,8-trimethyl-4H-[1,3]dioxino[4,5-c]pyridine-5-ylmethyl)-amide (19) (8.2 g, 19.8 mmol), following the procedure described in Example 4, gave 4′-cyano-biphenyl-4-carboxylic acid (5-hydroxy-4-hydroxymethyl-6-methyl-pyridin-3-ylmethyl)-amide (21) (7.0 g, 94% yield).

¹H-NMR (DMSO-d₆): δ 9.20 (s, 1H), 8.92 (t, 1H), 8.00-7.85 (m, 9H), 5.78 (s br, 1H), 4.78 (s, 2H), 4.50 (d, 2H), 2.34 (s, 3H).

### Example 22

### Synthesis of 4′-Cyano-biphenyl-4-carboxylic acid (4-hydroxymethyl-5-methoxy-6-methyl-pyridin-3-ylmethyl)-amide (22)

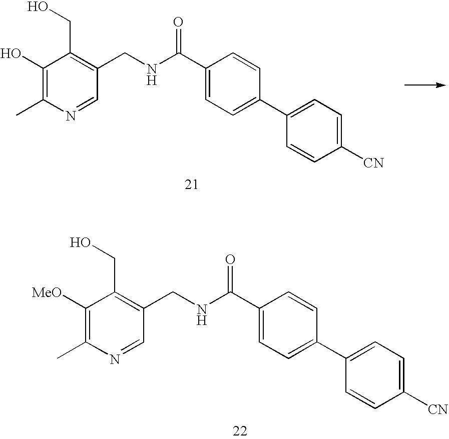

To a mixture of 4′-cyano-biphenyl-4-carboxylic acid (5-hydroxy-4-hydroxymethyl-6-methyl-pyridin-3-ylmethyl)-amide (213 mg, 0.57 mmol) and cesium carbonate (372 mg, 1.14 mmol) in dry acetonitrile was added methyl iodide (81 mg, 0.57 mmol) and the reaction was stirred overnight at room temperature. The mixture was filtered over a celite pad and washed several times with ethyl acetate. The filtrate was evaporated and the crude product was purified by column chromatography on silica gel to give 4′-cyano-biphenyl-4-carboxylic acid (4-hydroxymethyl-5-methoxy-6-methyl-pyridin-3-ylmethyl)-amide (22) (146 mg, 66%) as a colorless solid.

¹H-NMR (DMSO): δ 8.93 (t, 1H), 8.00-7.85 (m, 9H), 4.78 (s, 2H), 4.5 (d, 2H), 3.3 (s, 3H), 2.3 (s, 3H).

### Example 23

### Synthesis of 4′-Carbamimidoyl-biphenyl-4-carboxylic acid (4-hydroxymethyl-5-methoxy-6-methyl-pyridin-3-ylmethyl)-amide (23)

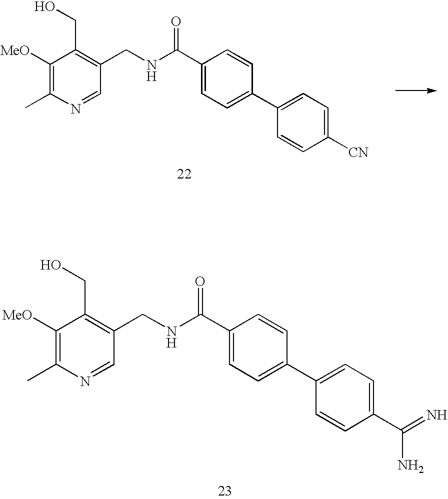

To a mixture of 4′-cyano-biphenyl-4-carboxylic acid (4-hydroxymethyl-5-methoxy-6-methyl-pyridin-3-ylmethyl)-amide (22) (30 mg, 0.08 mmol) in absolute ethyl alcohol, was bubbled anhydrous hydrogen chloride gas at 0° C. for 20 minutes, the reaction then sealed and stirred overnight. Removal of the solvent gave a light yellow solid. The resulting solid was dissolved in 7 N ammonia in methyl alcohol (10 mL) and stirred at 40° C. for overnight. After the solvent was removed and the mixture was purified by HPLC using a gradient mixture of 10%-100% methyl alcohol versus 0.1% trifluoroacetic acid in water, to give 4′-carbamimidoyl-biphenyl-4-carboxylic acid (4-hydroxymethyl-5-methoxy-6-methyl-pyridin-3-ylmethyl)-amide (23) (30 mg, 99% yield) as a yellow solid.

¹H-NMR (DMSO-d₆): δ 9.38-9.11 (2br, 3H), 8.29 (s, 1H), 8.04-7.93 (m, 8H), 4.70 (s, 2H), 4.68 (s, 2H), 3.80 (s, 3H), 2.50 (s, 3H).

### Example 24

### Synthesis of 5-{[(4′-Cyano-biphenyl-4 carbonyl)-amino]-methyl}-3-hydroxy-2-methyl-isonicotinic acid methyl ester (24)

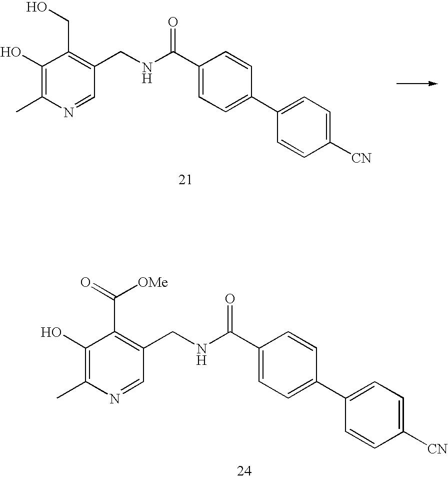

A mixture of 4′-cyano-biphenyl-4-carboxylic acid (5-hydroxy-4-hydroxymethyl-6-methyl-pyridin-3-ylmethyl)-amide (21) (2.05 g, 5.5 mmol), potassium cyanide (1.07 g, 16.4 mmol), manganese (IV) dioxide (5.73 g, 85%, 56.0 mmol), acetic acid (0.33 mL) and methyl alcohol (120 mL) was stirred at room temperature for 12 hours. The solid was filtered through a celite pad and washed several times with methanol. The solvent was evaporated and the crude residue purified on a silical gel column using ethyl acetate as an eluant to give 5-{[(4-cyano-biphenyl-4 carbonyl)-amino]-methyl}-3-hydroxy-2-methyl-isonicotinic acid methyl ester (24) (1.05 g, 48%) as a colorless solid.

¹H-NMR (DMSO-d₆): δ 8.94 (s br, 1H), 8.00-7.85 (m, 9H), 4.50 (s, 2H), 3.80 (s, 3H), 2.39 (s, 3H),

### Example 25

### Synthesis of 5-{[(4′-Carbamimidoyl-biphenyl-4-carbonyl)-amino]-methyl}-3-hydroxy-2-methyl-isonicotinic acid (25)

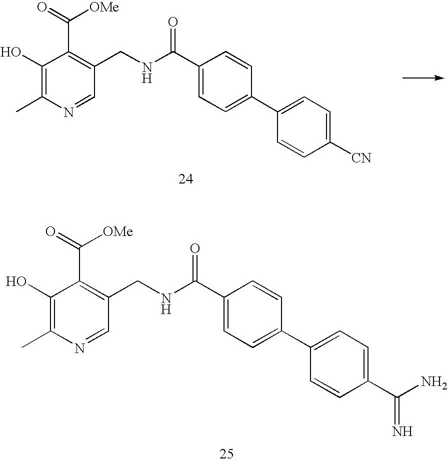

Hydrogen chloride gas was bubbled through a mixture of 5-{[(4-cyano-biphenyl-4-carbonyl)-amino]-methyl}-3-hydroxy-2-methyl-isonicotinic acid methyl ester (22) (137 mg, 0.34 mmol) in dry ethanol (4 mL) for 20 minutes at 0° C. The reaction mixture was then allowed to warm to room temperature and stirred overnight. The solvent was evaporated to give a yellowish residue which was then dissolved in 7 N ammonia methyl alcohol (10 mL) and stirred at 30° C. for 12 hours. Evaporation of the solvent gave a colorless solid that was then recrystallized from methanol to give 5-{[(4′-carbamimidoyl-biphenyl-4-carbonyl)-amino]-methyl}-3-hydroxy-2-methyl-isonicotinic acid (25) (112 mg, 79%).

¹H-NMR (DMSO-d₆): δ 9.04 (t, 1H), 7.97-7.73 (m, 9H), 7.13 (s, 1H), 4.53 (d, 2H), 3.16 (s, 3H), 2.17 (s, 3H).

### Example 26

### Synthesis of 5-{[(4′-Carbamimidoyl-biphenyl-4-carbonyl)-amino]-methyl}-3-hydroxy-2-methyl-isonicotinic acid (26)

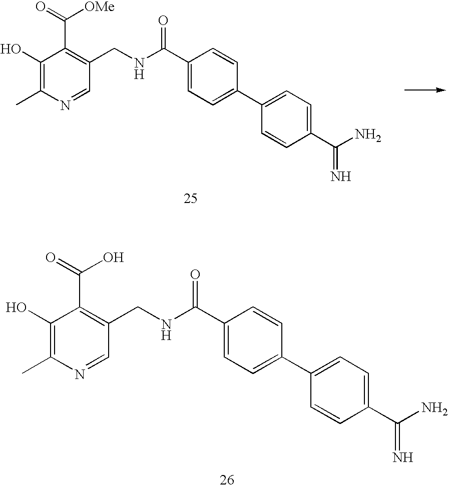

A mixture of 5-{[(4′-carbamimidoyl-biphenyl-4-carbonyl)-amino]-methyl}-3-hydroxy-2-methyl-isonicotinic acid (25) (32 mg, 0.08 mmol) in 4 N hydrochloric acid (3 mL) was refluxed for 25 minutes. The solvent was then removed and the residue was purified using HPLC with a solvent gradient of 10-100% methyl alcohol:trifluoroacetic acid mixture to obtain 5-{[(4′-carbamimidoyl-biphenyl-4-carbonyl)-amino]-methyl}-3-hydroxy-2-methyl-isonicotinic acid (26) (15 mg, 50% yield) as a colorless solid.

¹H-NMR (CD₃OD): δ 9.25-8.23 (br s, 3H), 8.16 (s, 1H), 7.83-8.02 (m, 8H), 4.70 (s, 2H), 2.61 (s, 3H).

### Example 27

### Synthesis of 5-{[(4′-Cyano-biphenyl-4-carbonyl)-amino]-methyl}-3-methoxy-2-methyl-isonicotinic acid methyl ester (27)

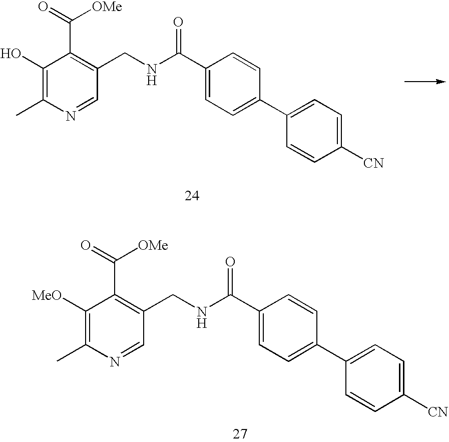

Methyl iodide (312 mg, 2.2 mmol) was added to a solution of 5-{[(4′-cyano-biphenyl-4-carbonyl)-amino]-methyl}-3-hydroxy-2-methyl-isonicotinic acid methyl ester (24) (440 mg, 1.10 mmol) and cesium carbonate (717 mg, 2.2 mmol) in dry acetone (20 mL). The mixture was stirred at room temperature for 12 hours in the absence of light. The mixture was then filtered, concentrated and purified by column chromatography using ethyl acetate:hexane (4:1), to give 5-{[(4′-cyano-biphenyl-4-carbonyl)-amino]methyl}-3-methoxy-2-methyl-isonicotinic acid methyl ester (27) (130 mg, 29% yield) as a colorless solid.

¹H-NMR (CDCl₃): δ 8.45 (s, 1H), 7.89-7.61 (m, 8H), 6.79 (br, 1H), 4.63 (d, 2H), 3.99 (s, 3H), 3.84 (s, 3H), 2.56 (s, 3H).

### Example 28

### Synthesis of 5-{[(4′-Carbamimidoyl-biphenyl-4-carbonyl)-amino]-methyl}-3-methoxy-2-methyl-isonicotinic acid methyl ester (28)

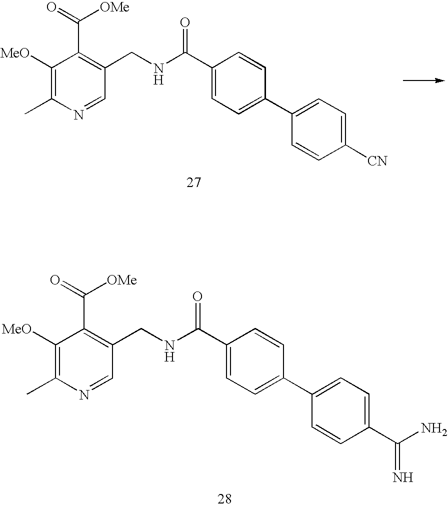

The conversion of nitrile (27) to amidine (28) was carried out as shown in Example 23.

¹H-NMR (DMSO): δ 9.07 (br, 1H), 8.31 (s, 1H), 8.03-7.92 (m, 8H), 4.49 (s, 2H), 3.84 (s, 3H), 3.74 (s, 3H), 2.45 (s, 3H).

### Example 29

### Synthesis of (5-Bromo-pyridin-2-yl)-(2,2,8-trimethyl-4H-[1,3]dioxino[4,5-c]pyridin-5-ylmethyl)-amine (29)

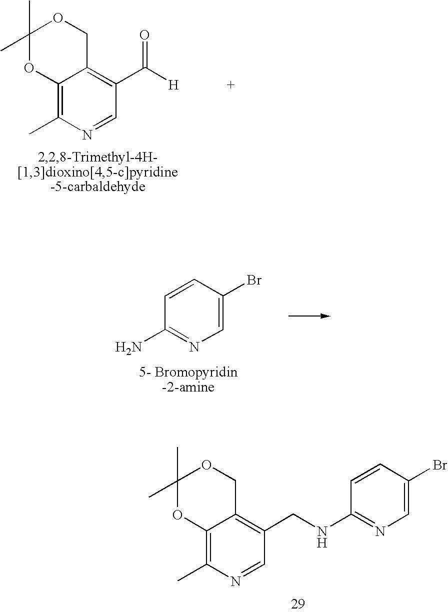

The reductive amination of 5-bromopyridine-2-amine (290 mg, 1.68 mmol) and 2,2,8-trimethyl-4H-[1,3]dioxino[4,5-c]pyridine-5-carbaldehyde (350 mg, 1.68 mmol), as described in Example 6, gave (5-bromo-pyridin-2-yl)-(2,2,8-trimethyl-4H-[1,3]dioxino[4,5-c]pyridin-5-ylmethyl)-amine (29) (176 mg, 29% yield) as a colorless solid.

¹H-NMR (CDCl₃): δ 8.13 (m, 1H), 8.05 (m, 1H), 7.50 (m, 1H), 6.37 (m, 1H), 4.93 (s, 2H), 4.40 (s, 2H), 2.46 (s, 3H), 1.57 (m, 7H).

### Example 30

### Synthesis of 4-{6-[(2,2,8-Trimethyl-4H-[1,3]dioxino[4,5-c]pyridin-5-ylmethyl)-amino]-pyridin-3-yl}-benzonitrile (30)

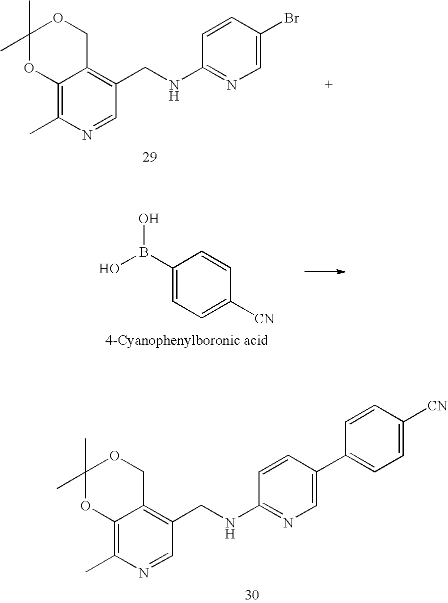

A mixture of (5-bromo-pyridin-2-yl)-(2,2,8-trimethyl-4H-[1,3]dioxino[4,5-c]pyridin-5-ylmethyl)-amine (29) (170 mg, 0.47 mmol), triphenylphosphine (54 mg, 0.05 mmol), and cesium carbonate (456 mg, 1.40 mmol) was stirred in toluene (30 mL) for 5 minutes. To the reaction mixture was added a solution of 4-cyanophenylboronic acid (68 mg, 0.47 mmol) in toluene (20 mL), followed by the addition of a mixture of iso-butyl alcohol and water (60 mL, 6:2). The reaction mixture was then heated at 80° C. for 5 hours, filtered through a celite pad and the pad washed with ethyl acetate (100 mL). The solvent was evaporated and the crude product was purified by column chromatography on silica gel using a mixture of dichloromethane:methyl alcohol (5:1) as eluant to give 4-{6-[(2,2,8-trimethyl-4H-[1,3]dioxino[,5-c]pyridin-5-ylmethyl)-amino]-pyridin-3-yl}-benzonitrile (30) (103 mg, 57% yield) as a light yellow solid.

¹H-NMR (CDCl₃): δ 8.38 (s, 1H), 8.07 (s 1H), 7.80-7.50 (m, 6H), 6.54 (d, 1H), 4.95 (s, 2H), 4.49 (s, 2H), 2.45 (s, 3H), 1.57 (s, 6H). ¹H-NMR (DMSO-_{d}): δ 8.30 (s, 1H), 7.82 (s, 1H), 7.75-7.60 (m, 2H), 7.55-7.38 (m, 2H), 7.24-7.15 (m, 1H), 6.55-6.48 (m, 1H), 4.80 (s, 2H), 4.26 (d, 2H), 2.13 (s, 3H), 1.37 (m, 6H).

### Example 31

### Synthesis of 4-{6-[(5-Hydroxy-4-hydroxymethyl-6-methyl-pyridin-3-ylmethyl)-amino]-pyridin-3-yl}-benzamidine (31)

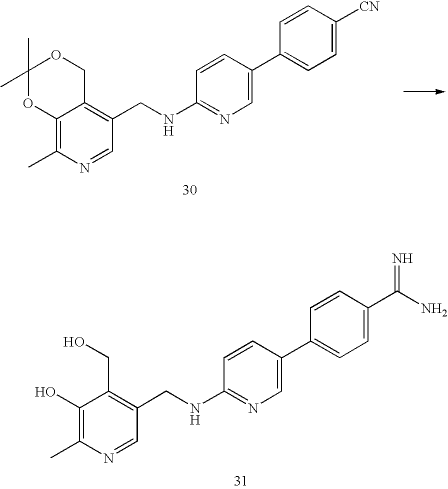

The conversion nitrile (30) to amidine (31) was carried out as shown in Example 2.

¹H-NMR (DMSO-d₆): δ 9.30-8.70 (m, 6H), 8.23 (s, 1H), 7.70-7.50 (m, 6H), 6.54 (d, 1H), 4.53 (s, 2H), 4.33 (d, 2H), 2.09 (s, 3H).

### Example 32

### Synthesis of 4′-[(2,2,8-Trimethyl-4H-[1,3]dioxino[4,5-c]pyridin-5-ylmethyl)-amino]-3′-fluoro-biphenyl-4-carbonitrile (32)

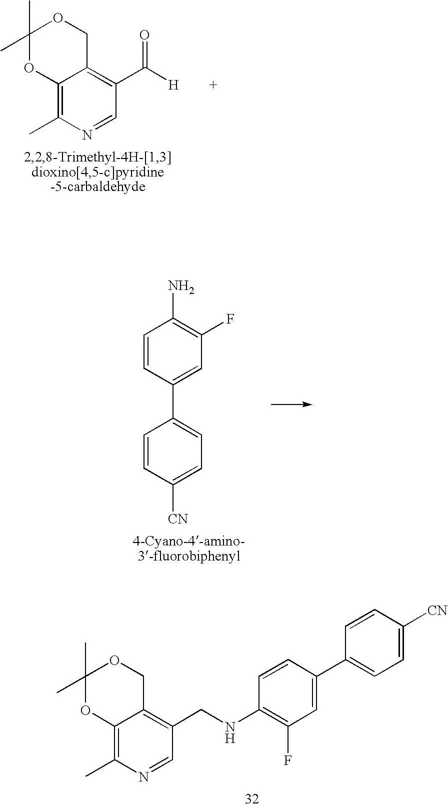

The reductive amination of 4-cyano-4′-amino-3′-fluorobiphenyl (400 mg, 1.88 mmol) and 2,2,8-trimethyl-4H-[1,3]dioxino[4,5-c]pyridine-5-carbaldehyde (390 mg, 1.88 mmol), as described in Example 6, gave 4′-[(2,2,8-trimethyl-4H-[1,3]dioxino[4,5-c]pyridin-5-ylmethyl)-amino]-3′-fluoro-biphenyl-4-carbonitrile (32) (356 mg, 47% yield) as a colorless solid.

¹H-NMR (DMSO-d₆): δ 7.90 (m, 1H), 7.79 (m, 4H), 7.55 (m, 1H), 7.39 (m, 1H), 6.75 (m, 1H), δ 6.44 (m, 1H), 4.93 (s, 2H), 4.30 (s, 2H), 2.25 (s, 3H), 1.48 (m, 6H). ¹⁹F-NMR decoupled (DMSO-d₆): δ −133.99 (s).

### Example 33

### Synthesis of 4′-[(5-Hydroxy-4-hydroxymethyl-6-methyl-pyridine-3-ylmethyl)-amino]-3′-fluoro-biphenyl-4-carbonitrile (33)

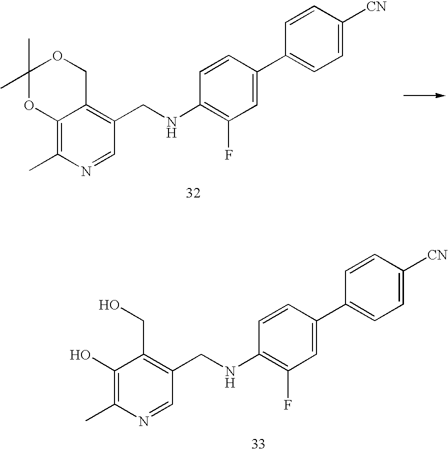

The hydrolysis of 3′-fluoro-4′-[(2,2,8-trimethyl-4H-[1,3]dioxino[4,5-c]pyridin-5-ylmethyl)-amino]-biphenyl-4-carbonitrile (32) (352 mg, 0.87 mmol) to 4′-[(5-hydroxy-4-hydroxymethyl-6-methyl-pyridine-3-ylmethyl)-amino]-3′-fluoro-biphenyl-4-carbonitrile (33) (254 mg, 80% yield) was carried out as described in Example 4.

¹H-NMR (DMSO-d₆): δ 7.98 (m, 1H), 7.92 (m, 4H), 7.68 (m, 1H), 7.53 (m, 1H), 6.89 (m, 1H), 6.47 (m, 1H), 4.88 (s, 2H), 4.54 (s, 2H), 2.44 (s, 3H). ¹⁹F-NMR decoupled (DMSO): δ −138.22(s).

### Example 34

### Synthesis of 3′-Trifluoromethoxy-4′-[(2,2,8-trimethyl-4H-[1,3]dioxino[4,5,c]pyridin-5-ylmethyl)-amino]-biphenyl-carbonitrile (34)

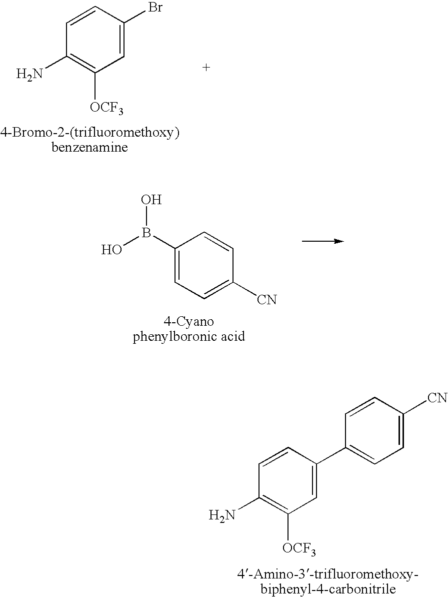

Step 1: A mixture of 4-bromo-2-(trifluoromethoxy)benzenamine (512 mg, 2.0 mmol), 4-cyanophenylboronic acid (324 mg, 2.2 mmol), 5% activated palladium on carbon (50% wet, 100 mg) and sodium carbonate (424 mg, 4.0 mmol) in a mixture of methanol:water (20 mL, 1:1) was heated at 70° C. for 12 hours. The reaction mixture was filtered through a celite pad and the filtrate evaporated to give a crude residue. Purification on silica gel using a mixture of ethyl acetate:hexane (4:1) as eluant gave the light yellow solid 4-amino-3-trifluoromethoxy-biphenyl-4-carbonitrile (210 mg, 38% yield).

¹H-NMR (CDCl₃): δ 7.58-7.70 (m, 4H), 7.33-7.39 (m, 2H), 6.88 (d, 1H), 4.06 (s br, 2H). ¹⁹F-NMR decoupled (CDCl₃): δ−58.15 (s).

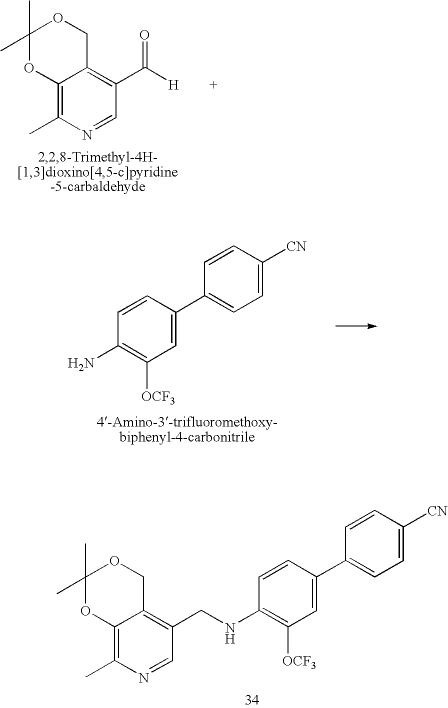

Step 2: The reductive amination of 4′-amino-3′-trifluoromethoxy-biphenyl-4-carbonitrile (210 mg, 0.75 mmol) and 2,2,8-trimethyl-4H-[1,3]dioxino[4,5-c]pyridine-5-carbaldehyde (186 mg, 0.90 mmol), as described in Example 6, gave 3-trifluoromethoxy-4-[(2,2,8-trimethyl-4H-[1,3]dioxino[4,5,c]pyridin-5-ylmethyl)-amino]-biphenyl-carbonitrile (34).

¹H-NMR (CDCl₃): δ 8.05 (s, 1H), 7.70-7.42 (m, 6H), 6.82 (d, 1H), 4.93 (s, 2H), 4.27 (s, 2H), 2.43 (s, 3H)

### Example 35

### Synthesis of 4-[(5-Hydroxy-4-hydroxymethyl-6-methyl-pyridin-3ylmethyl)-amino]-3-trifluoromethoxy-biphenyl-4-carboxamidine (35)

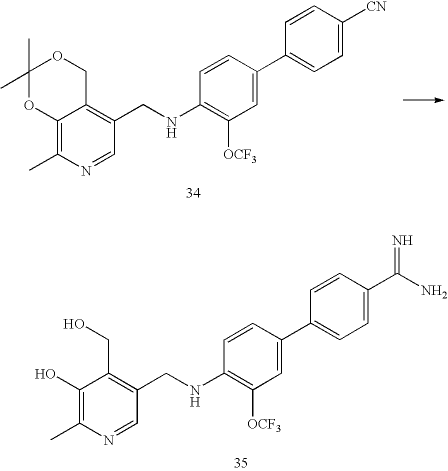

The conversion of nitrile (34) to amidine (35) was carried out as shown in Example 23.

¹H-NMR (DMSO-d₆): δ 8.96-9.27 (2 br, 3H), 7.90 (s, 1H), 7.85 (s, 4H), 7.66 (s, 1H), 7.59 (d, 1H), 6.79 (d, 2H), 4.90 (s, 2H), 4.63 (br s, 2H), 2.51 (s, 3H). ¹⁹F-NMR decoupled (CDCl₃): δ−74.51 (s).

### Example 36

### Synthesis of 3′-Trifluoromethyl-4′-[(2,2,8-trimethyl-4H-[1,3]dioxino[4,5-c]pyridin-5-ylmethyl)-amino]-biphenyl-4-carbonitrile (36)

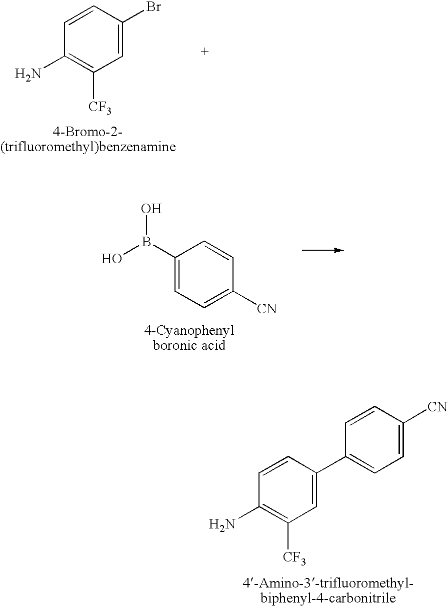

To a solution of 4-bromo-2-(trifluoromethyl)benzenamine (309 mg, 2.1 mmol) in a 1:1 mixture of methyl alcohol:water (20 mL) was added solid sodium carbonate (424 mg, 4.0 mmol), followed by 4-cyanophenyl boronic acid (324 mg, 2.2 mmol) and 5% activated palladium on carbon (50% wet, 100 mg). The reaction mixture was heated at 75° C. for 12 hours, then filtered through a celite pad and the residue washed with hot methanol. The solvent was evaporated and the mixture purified by silica gel column chromatography using acetate:hexane (4:1) as eluant to give 4′-amino-3′-trifluoromethyl-biphenyl-4-carbonitrile (97 mg, 19% yield) as a light yellow solid.

¹H-NMR (CDCl₃): δ 7.70-7.53 (m, 6H), 6.84 (d, 1H), 4.35 (s, 2H). ¹⁹F-NMR (CDCl₃): δ −63.28 (s).

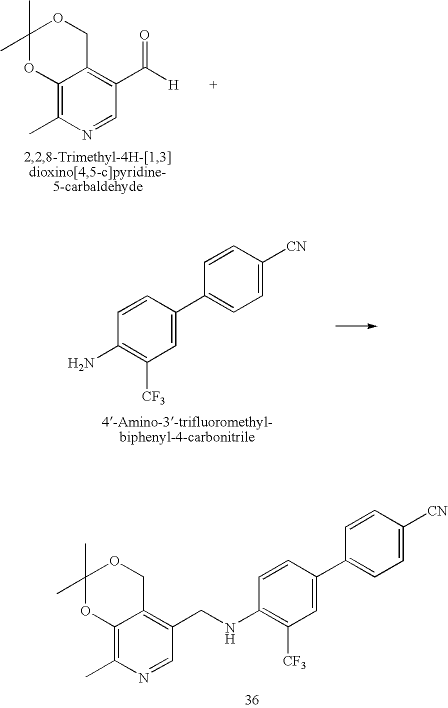

The reductive amination of 4′-amino-3′-trifluoromethyl-biphenyl-4-carbonitrile (95 mg, 0.36 mmol) and the 2,2,8-trimethyl-4H-[1,3]dioxino[4,5-c]pyridine-5-carbaldehyde (75 mg, 0.36 mmol), as described in Example 6, gave the light yellow solid 3′-trifluoromethyl-4′-[(2,2,8-trimethyl-4H-[1,3]dioxino[4,5-c]pyridin-5-ylmethyl)-amino]-biphenyl-4-carbonitrile (36) (70 mg, 40% yield).

¹H NMR (CDCl₃): δ 8.06 (s, 1H), 7.72-7.60 (m, 6H), 6.86 (d, 1H), 4.87 (s, 2H), 4.57 (s, 1H), 4.31 (s, 2H), 2.43 (s, 3H), 1.56 (s, 6H). ¹⁹F-NMR (CDCl₃): δ −62.0 (s).

### Example 37

### Synthesis of 4′-[(5-Hydroxy-4-hydroxymethyl-6-methyl-pyridin-3-ylmethyl)-amino]-3′-trifluoromethyl-biphenyl-4-carboxamidine (37)

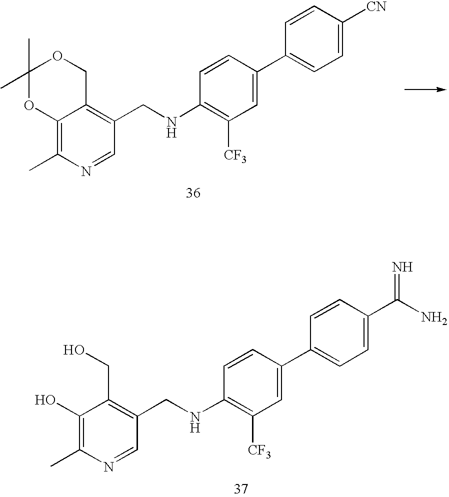

The conversion nitrile (36) to amidine (37) was carried out as described in Example 23.

¹H-NMR (DMSO-d₆): δ 8.80 (br s, 3H), 7.91-7.77 (m, 7H), 6.85 (d, 1H), 6.44 (t, 1H), 4.76 (s, 2H), 4.56 (d, 2H), 2.31 (s, 3H). ¹⁹F-NMR (DMSO-d₆): δ −61.7 (s).

### Example 38

### Synthesis of 4-[(3-Hydroxy-5-hydroxymethyl-2-methyl-pyridin-4-ylmethyl)-amino]-benzonitrile (38)

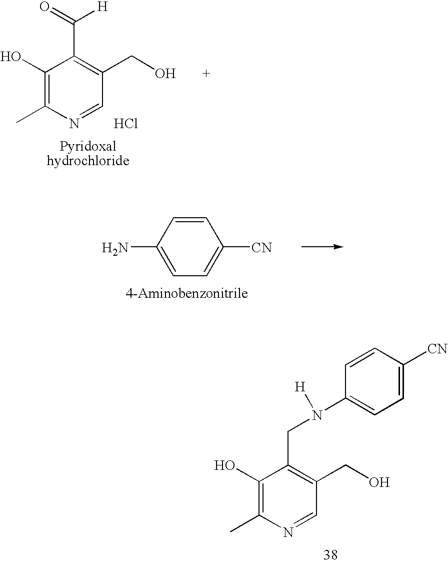

The reductive amination of pyridoxal hydrochloride (2.04 g, 10.0 mmol) and 4-aminobenzonitrile (1.3 g, 11.0 mmol), as described in Example 6, gave 4-[(3-hydroxy-5-hydroxymethyl-2-methyl-pyridin-4-ylmethyl)-amino]-benzonitrile (38) (1.30 g, 48% yield) as a colorless solid.

¹H-NMR (CD₃OD): δ 7.93 (s, 1H), 7.42 (d, 2H), 6.78 (d, 2H), 4.70 (s, 2H), 4.51 (s, 2H), 2.47 (s, 3H).

### Example 39

### Synthesis of 4-[(3-Hydroxy-5-hydroxymethyl-2-methyl-pyridin-4-ylmethyl)-amino]-benzamidine (39)

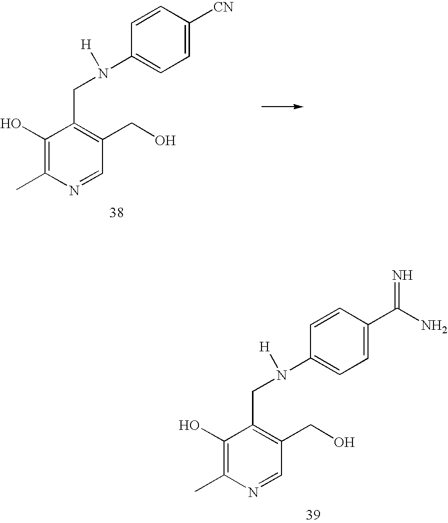

The conversion of nitrile (38) to amidine (39) was carried out as described in Example 23.

¹H-NMR (DMSO-d₆): δ 7.60 (d, 2H), 7.40 (s, 1H), 6.66 (d, 2H), 4.43 (s, 2H), 4.29 (s, 2H), 2.25 (s, 3H). MS m/z (ES⁺): 287.15 (M+H⁺).

### Example 40

### Synthesis of 4′-[(3-Hydroxy-5-hydroxymethyl-2-methyl-pyridin-4-ylmethyl)-amino]-biphenyl-4-carboxamidine (41)

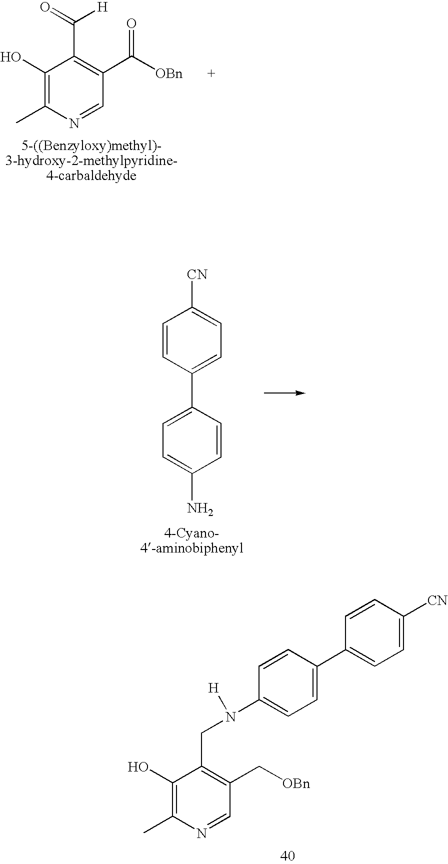

Step 1: The reductive amination of 5-((benzyloxy)methyl)-3-hydroxy-2-methylpyridine-4-carbaldehyde (425 mg, 1.64 mmol) and 4-cyano-4′-aminobiphenyl (342 mg, 1.76 mmol), using the procedure described in Example 6, gave 4′-[(5-benzyloxymethyl-3-hydroxy-2-methyl-pyridin-4-ylmethyl)-amino]-biphenyl-4-carbonitrile (40) (228 mg, 60% yield) as a light yellow solid.

¹H-NMR (CDCl₃): δ 7.99 (s, 1H), 7.72-7.65 (m, 2H), 7.63-7.58 (m, 2H), 7.48-7.40 (m, 2H), 7.34-7.27 (m, 5H), 6.93-6.85 (m, 2H), 4.57 (s, 2H), 4.57 (s, 2H), 4.54 (s, 2H), 2.45 (s, 3H). MS. (M+1, ESI): 436.4 and (M+Na, ESI): 458.3.

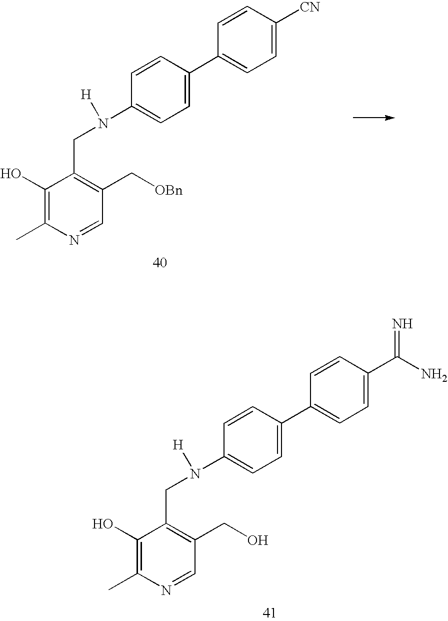

Step 2: The conversion of nitrile (40) to amidine (41) was carried out as described in Example 2.

¹H-NMR (CD₃OD): δ 8.00 (s, 1H), 7.90-7.78 (m, 4H), 7.65-7.55 (m, 2H), 6.97-6.87 (m, 2H), 4.78 (s, 2H), 4.61 (s, 2H), 2.50 (s, 3H). MS. m/z (ES⁺): 363.45 (M+H⁺).

### Example 41

### Synthesis of 4′-[(5-Hydroxy-4,6-dimethyl-pyridin-3-ylmethyl)-amino]-biphenyl-4-carboxamidine (43)

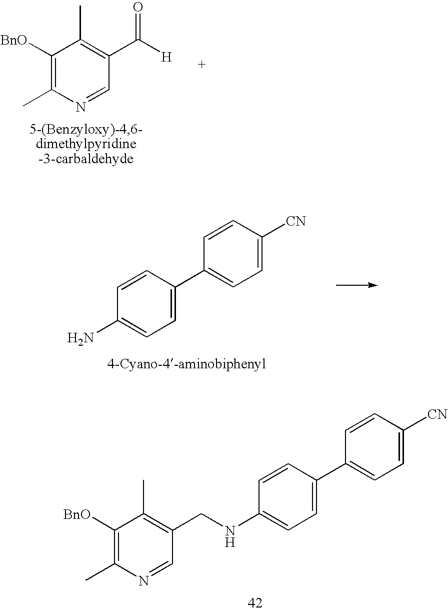

Step 1: The reductive amination of 5-(benzyloxy)-4,6-dimethylpyridine-3-carbaldehyde (500 mg, 2.1 mmol) and 4-cyano-4′-aminobiphenyl (486 mg, 2.5 mmol), using the procedure described in Example 6, gave 4-[(5-benzyloxy-4,6-dimethyl-pyridin-3-ylmethyl)-amino]-biphenyl-4-carbonitrile (42) (300 mg, 34% yield) as a light yellow solid.

¹H-NMR (CDCl₃): δ 8.24 (s, 1H), 7.64 (m, 4H), 7.44 (m, 7H), 6.74 (d, 2H), 4.84 (s, 2H), 4.31 (d, 2H), 4.98 (s, 1H), 2.64 (s, 3H) & 2.30 (s, 3H)

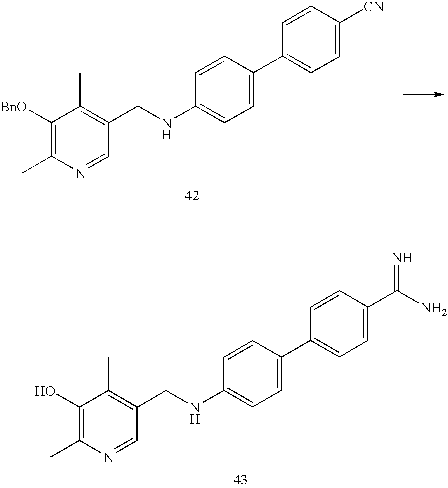

Step 2: The conversion of nitrile (42) to amidine (43) was carried out as described in Example 2.

¹H-NMR (CD₃OD): δ 7.90-7.75 (m, 5H), 7.56 (d, 2H), 6.76 (d, 2H), 4.40 (s, 2H), 2.48 (s, 3H,), 2.37 (s, 3H)

### Example 42

### Synthesis of N-[5-(4-tert-Butyl-benzyloxy)-4-hydroxymethyl-6-methyl-pyridin-3-ylmethyl]-4-cyano-benzamide (44)

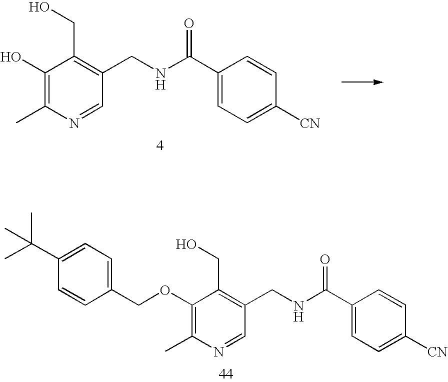

A mixture of compound 4 (300 mg, 1 mmol), 4-(tert-butyl) benzyl chloride (0.5 mL) and cesium carbonate (493 mg, 1.5 mmol) in anhydrous DMF (10 mL) was stirred for 2.5 hours at room temperature. The solvent was evaporated, and the crude mixture was purified by column chromatography on silica gel column using a mixture of dichloromethane:methyl alcohol (15:1) as eluant to give N-[5-(4-tert-butyl-benzyloxy)-4-hydroxymethyl-6-methyl-pyridin-3-ylmethyl]-4-cyano-benzamide (44) (547 mg, 82% yield) as a colorless solid.

¹H-NMR (DMSO-d₆): δ 8.35 (m, 1H), 7.85-7.82 (m, 2H), 7.69-7.67 (m, 2H), 7.44-7.42 (m, 3H), 7.35-7.32 (m, 2H), 7.26 (m, 1H), 4.91 (s, 2H), 4.68-4.66 (s, 2H), 2.59 (s, 3H), 1.32 (s, 9H).

### Example 43

### Synthesis of (R)-3-(4-Cyano-phenyl)-3-[(2,2,8-trimethyl-4H-[1,3]dioxino[4,5-c]pyridin-5-ylmethyl)-amino]-propionic acid (45)

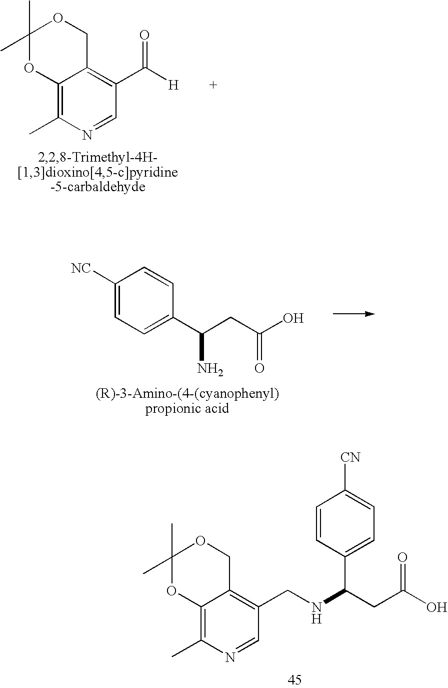

A mixture of (R)-3-amino-(4-(cyanophenyl) propionic acid (470 mg, 2.45 mmol), and 2,2,8-trimethyl-4H-[1,3]dioxino[4,5-c]pyridine-5-carbaldehyde (680 mg, 3.28 mmol) in methyl alcohol (50 mL) was refluxed under nitrogen for 2 hours. The reaction mixture was allowed to cool to room temperature, then sodium borohydride (1.00 g, 26 mmol) was added and the reaction was stirred at room temperature for 12 hours. The solvent was evaporated to leave a crude solid which was purified by column chromatography over silica gel using 10% methyl alcohol in dichloromethane, followed by dichloromethane:methyl alcohol:ammonia in water (10:5:1) to give (R)-3-(4-cyano-phenyl)-3-[(5-hydroxy-4-hydroxymethyl-6-methyl-pyridin-3-ylmethyl)-amino]-propionic acid (45) (500 mg, 53% yield) as a colorless solid.

¹H-NMR (CD₃OD): δ 7.74 (s, 1H), 7.70 (d, 2H), 7.58 (d, 2H), 4.88 (q, 2H), 4.18 (dd, 1H), 3.59-3.49 (m, 2H), 2.63 (dd, 1H), 2.50 (dd, 1H), 2.31 (s, 3H), 1.51 (6H).

### Example 44

### Synthesis of (R)-3-(4-Cyano-phenyl)-3-[(5-hydroxy-4-hydroxymethyl-6-methyl-pyridin-3-ylmethyl)-amino]-propionic acid (46)

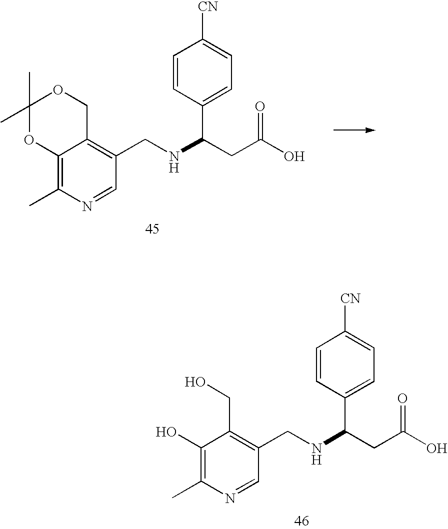

A mixture of (R)-3-(4-cyano-phenyl)-3-[(5-hydroxy-4-hydroxymethyl-6-methyl-pyridin-3-ylmethyl)-amino]-propionic acid (45) (300 mg, 0.87 mmol), α-bromo-4-tolunitrile (256 mg, 2.17 mmol) and cesium carbonate (600 mg, 1.84 mmol) in anhydrous DMF (50 mL) was stirred for 12 hours. Removal of solvent gave a crude residue which was purified by column chromatography on silica gel using 10% methyl alcohol in dichloromethane as eluant to give (R)-3-(4-cyano-phenyl)-3-[(5-hydroxy-4-hydroxymethyl-6-methyl-pyridin-3-ylmethyl)-amino]-propionic acid (46) (150 mg, 38% yield).

¹H-NMR (CDCl₃): δ 8.02 (s, 1H), 7.68-7.60 (m. 4H), 7.57 (d, 2H), 7.50 (d, 2H), 4.97 (dd, 2H), 4.66 (d, 1H), 4.44 (d, 1H), 4.21 (dd, 1H), 3.6 (d, 2H), 2.7 (dd, 2H), 2.46 (s, 3H).

### Example 45

### Synthesis of (S)-3-(4-Cyano-phenyl)-3-[(2,2,8-trimethyl-4H-[1,3]dioxino[4,5-c]pyridin-5-ylmethyl)-amino]-propionic acid (55)

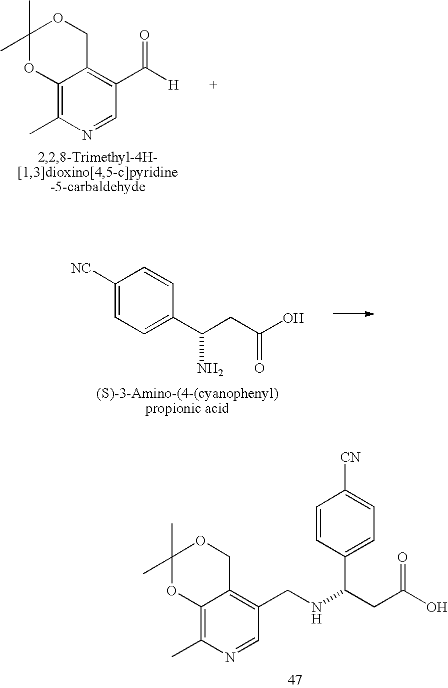

A mixture of (S)-3-amino-(4-(cyanophenyl) propionic acid (846 mg, 4.42 mmol), and 2,2,8-trimethyl-4H-[1,3]dioxino[4,5-c]pyridine-5-carbaldehyde (1.2 g, 5.8 mmol) in methyl alcohol (30 mL) was refluxed under nitrogen for 2 hours. The reaction mixture was allowed to cool to room temperature. Sodium borohydride (1.0 g, 26.4 mmol) was then added and the reaction stirred at room temperature for 12 hours. Removal of solvent gave a crude residue which was purified by column chromatography on silica gel using a mixture of dichloromethane:methyl alcohol:ammonium hydroxide (12:6:1) as eluant to give (S)-3-(4-cyano-phenyl)-3-[(5-hydroxy-4-hydroxymethyl-6-methyl-pyridin-3-ylmethyl)-amino]-propionic acid (47) (0.8 g, 35% yield) as a colorless solid.

¹H-NMR (CD₃OD): δ 7.74 (s, 1H), 7.67 (d, 2H), 7.58 (d, 2H), 4.88 (q, 2H), 4.18 (dd, 1H), 3.59-3.49 (m, 2H), 2.63 (dd, 1H), 2.50 (dd, 1H), 2.27 (s, 3H), 1.51 (s, 6H).

### Example 46

### Synthesis of (S)-3-(4-Cyano-phenyl)-3-[(5-hydroxy-4-hydroxymethyl-6-methyl-pyridin-3-ylmethyl)-amino]-propionic acid (48)

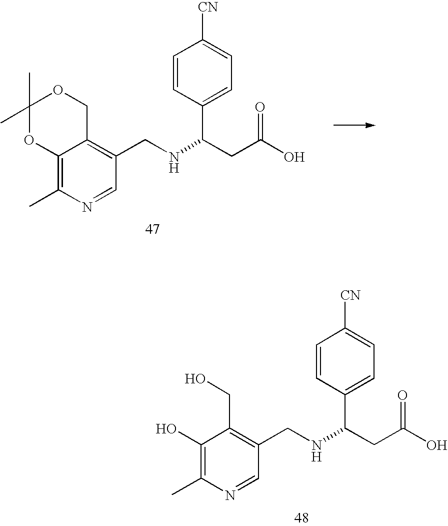

(S)-3-(4-Cyano-phenyl)-3-[(2,2,8-trimethyl-4H-[1,3]dioxino[4,5-c]pyridin-5-ylmethyl)-amino]-propionic acid (47) (0.75 g, 2.0 mmol) was stirred in a solution of 20% formic acid in water (100 mL) at room temperature for 5 days. Removal of solvent gave (S)-3-(4-cyano-phenyl)-3-[(5-hydroxy-4-hydroxymethyl-6-methyl-pyridin-3-ylmethyl)-amino]-propionic acid (48) (0.5 g, 73% yield) as a colorless solid.

¹H-NMR (CD₃OD): δ 7.91, (s, 1H), 7.79 (d, 2H), 7.71 (d, 2H), 5.34 (s, 2H), 4.90 (d, 2H), 4.69-4.61 (m, 1H), 4.07 (q, 2H), 3.03 (dd, 1H), 2.87 (dd, 1H), 2.46 (s, 3H).

### Example 47

### Synthesis of 3-(N-(4-Cyanobenzyl)-N-((2,2,8-trimethyl-4H-[1,3]dioxino[4,5-c]pyridin-5-yl)methyl)amino)benzonitrile (50)

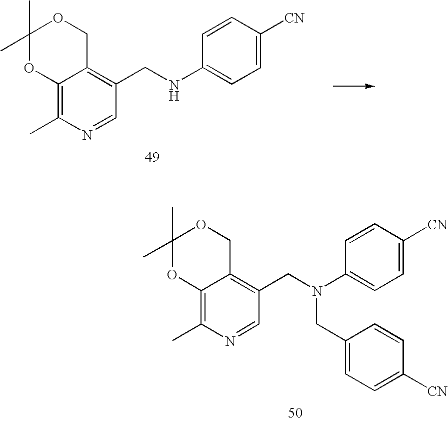

A solution of 4-((2,2,8-trimethyl-4H-[1,3]dioxino[4,5-c]pyridin-5-yl)methylamino) benzonitrile (49) (3.09 g, 10 mmol) in anhydrous DMF (10 mL) was added to a suspension of sodium hydride (60% in mineral oil, 800 mg, 20 mmol) in anhydrous DMF (100 mL) at 0° C., followed by the addition of 4-cyanobenzylbromide (2.16 g, 11 mmol). The solvent was evaporated, diluted with water and extracted with dichloromethane. The organic layer was dried over magnesium sulfate, filtered and evaporated to give the crude sample, which was purified by column chromatography on silica gel using mixture of ethyl acetate:hexane (1:2 to 1:1) as eluant to give 3-(N-(4-cyanobenzyl)-N-((2,2,8-trimethyl-4H-[1,3]dioxino[4,5-c]pyridin-5-yl)methyl)amino)benzonitrile (50) (2.70 g, 64% yield) as a light yellow solid.

¹H-NMR (CDCl₃): δ 7.78 (s, 1H), 7.64 (d, 2H), 7.46 (d, 2H), 7.27 (d, 2H), 6.67 (d, 2H), 4.71 (s, 2H), 4.46 (s, 2H), 2.40 (s, 3H), 1.55 (s, 6H).

### Example 48

### Synthesis of 4-(N-(4-Cyanobenzyl)-N-((5-hydroxy-4-(hydroxymethyl)-6-methylpyridin-3-yl)methyl)amino)benzonitrile (51)

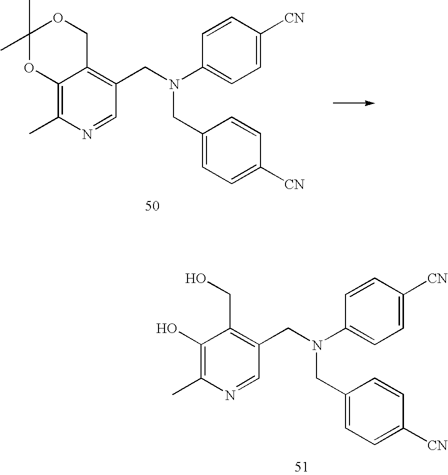

The hydrolysis of (50) gave (51) was carried out as described in Example 4.

¹H-NMR (DMSO-d₆): δ 7.84-6.72 (m, 9H), 4.87 (s, 4H), 4.69 (s, 2H), 2.33 (s, 3H).

### Example 49

### Synthesis of 4-(N-(4-Carbamimidoyl-benzyl)-N-((5-hydroxy-4-(hydroxymethyl)-6-methylpyridin-3-yl)methyl)amino)benzamidine (52)

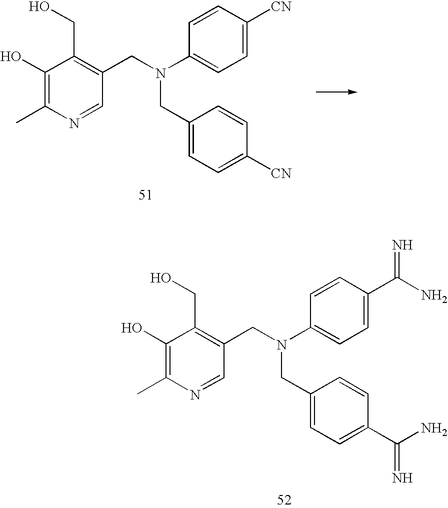

The conversion of nitrile (51) to amidine (52) was carried out as described in Example 23.

¹H-NMR (DMSO-d₆): δ 9.28-8.87 (m, 6H), 7.82-6.79 (m, 9H), 5.06 (s, 2H), 4.99 (s, 2H), 2.51 (s, 3H).

### Example 50

### Synthesis of 2-(2,2,8-Trimethyl-4H-[1,3]dioxino[4,5-c]pyridin-5-yl)ethanamine (53)

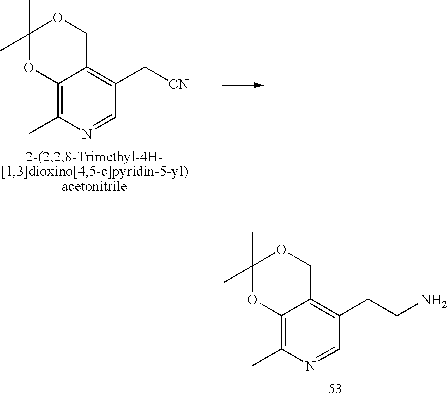

2-(2,2,8-Trimethyl-4H-[1,3]dioxino[4,5c]pyridin-5-yl)acetonitrile (8.72 g, 40.0 mmol) was added to a suspension of lithium aluminum hydride (6.08 g, 160 mmol) in anhydrous ethyl ether (350 mL) at 0° C. The reaction mixture was stirred at 0° C. for 1 hour and then at room temperature for overnight. Water was added slowly to destroy the excess of lithium aluminum hydride. The mixture was then filtered and the cake washed with ethyl acetate. The organic layer was dried over anhydrous magnesium sulfate, filtered and evaporated to give a crude mixture, then purified by column chromatography on silica gel using a mixture of dichloromethane:methyl alcohol:2 M ammonia in methyl alcohol (30:2:1 to 15:2:1) as eluant to obtain 2-(2,2,8-trimethyl-4H-[1,3]dioxino[4,5-c]pyridin-5-yl)ethanamine (53) (3.32 g, 37% yield) as a light yellow syrup.

¹H NMR (CDCl₃): δ 7.87 (s, 1H), 4.81 (s, 2H), 2.92 (t, 2H), 2.57 (t, 2H), 2.36 (s, 3H), 1.84 (s, 2H), 1.53 (s, 6H).

### Example 51

### Synthesis of 4-{[2-(2,2,8-Trimethyl-4H-[1,3]dioxino[4,5-c]pyridin-5-yl)-ethylamino]-methyl}-benzonitrile (54)+

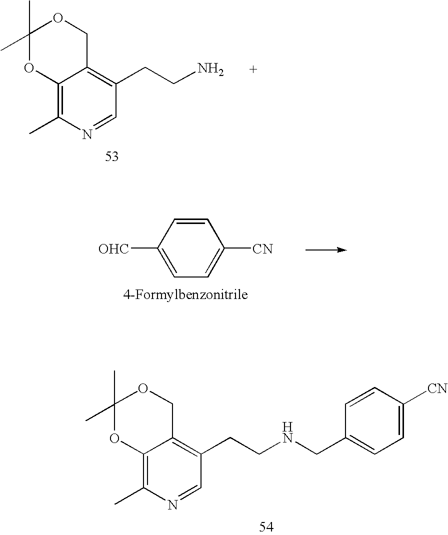

The reductive amination of 2-(2,2,8-trimethyl-4H-[1,3]dioxino[4,5-c]pyridin-5-yl)ethanamine (53) (444 mg, 2.0 mmol) and 4-formylbenzonitrile (262 mg, 2.0 mmol), following the procedure described in Example 6, gave 4-{[2-(2,2,8-trimethyl-4H-[1,3]dioxino[4,5-c]pyridin-5-yl)-ethylamino]-methyl}-benzonitrile (54) (318 mg, 47% yield) as a colorless solid.

¹H NMR (CDCl₃): δ 7.89 (s, 1H), 7.62 (d, 2H), 7.40 (d, 2H), 4.80 (s, 2H), 3.85 (s, 2H), 2.84 (t, 2H), 2.63 (t, 2H), 2.38 (s, 3H), 1.53 (s, 6H).

### Example 52

### Synthesis of 4-{[2-(5-Hydroxy-4-hydroxymethyl-6-methyl-pyridin-3-yl)-ethylamino]-methyl}-benzamidine (55)

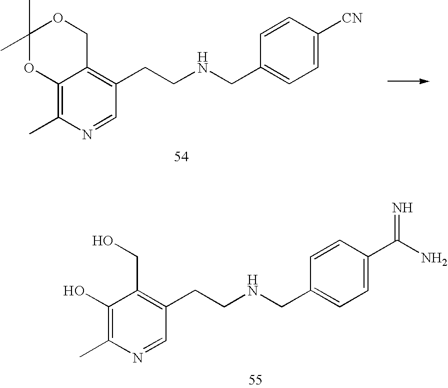

The conversion of nitrile (54) to amidine (55) was carried out as described in Example 2.

¹H-NMR (DMSO-d₆): δ 7.71 (s, 1H), 7.68 (s, 2H), 7.39 (d, 2H), 4.62 (s, 2H), 3.74 (s, 2H), 2.62-2.69 (m, 4H), 2.28 (s, 3H).

### Example 53

### Synthesis of 3-{[2-(2,2,8-Trimethyl-4H-[1,3]dioxino[4,5-c]pyridin-5-yl)-ethylamino]-methyl}-benzonitrile (56)

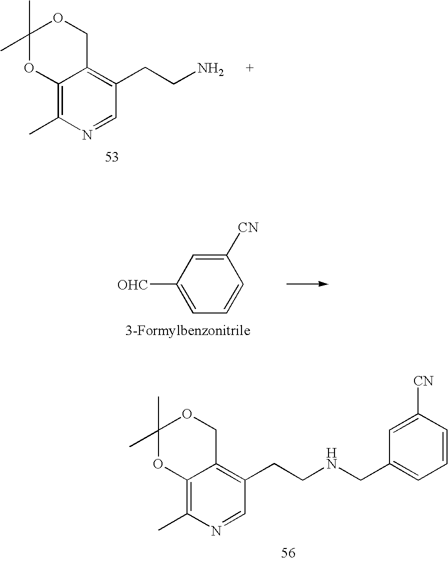

The reductive amination of 2-(2,2,8-trimethyl-4H-[1,3]dioxino[4,5-c]pyridin-5-yl)ethanamine (53) (444 mg, 2.0 mmol) and 3-formylbenzonitrile (262 mg, 2.0 mmol), following the procedure described in Example 6, gave 3-{[2-(2,2,8-trimethyl-4H-[1,3]dioxino[4,5-c]pyridin-5-yl)-ethylamino]-methyl}-benzonitrile (56) (218 mg, 32% yield) as a yellow syrup.

¹H-NMR (CDCl₃): δ 7.89 (s, 1H), 7.61-7.38 (m, 4H), 4.81 (s, 2H), 3.82 (s, 2H), 2.84 (t, 2H), 2.64 (t, 2H), 2.38 (s, 3H), 1.54 (s, 6H).

### Example 54

### Synthesis of 3-{[2-(5-Hydroxy-4-hydroxymethyl-6-methyl-pyridin-3-yl)ethylamino]methyl}-benzamidine (57)

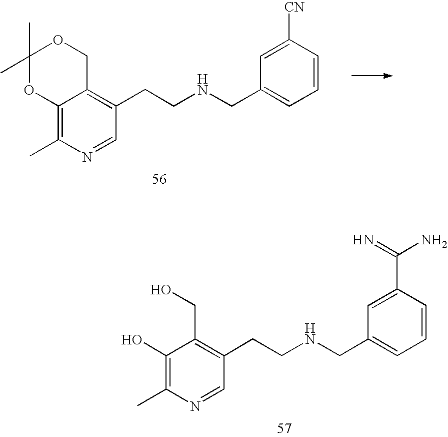

The conversion of nitrile (56) to amidine (57) was carried out as described in Example 2.

¹H-NMR (DMSO-d₆): δ 7.70 (s, 1H), 7.69 (s, 1H), 7.62-7.39 (m, 5H), 4.63 (s, 2H), 3.73 (s, 2H), 3.00-3.00 (s br, 1H), 2.69-2.67 (m, 4H), 2.28 (s, 3H).

### Example 55

### Synthesis of 4-Cyano-N-[2-(2,2,8-trimethyl-4H-[1,3]dioxino[4,5-c]pyridin-5-yl)-ethyl]-benzamide (58)

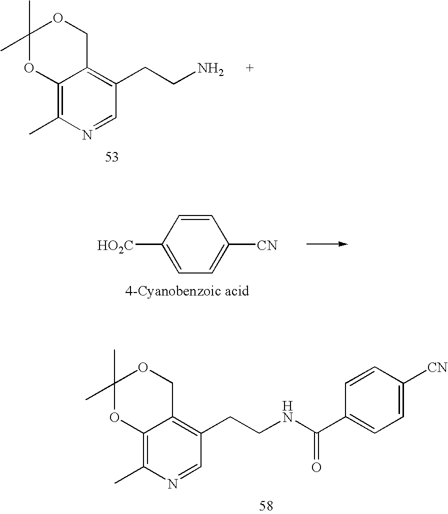

The coupling of 2-(2,2,8-trimethyl-4H-[1,3]dioxino[4,5-c]pyridin-5-yl)ethanamine (53) (444 mg, 2.0 mmol) and 4-cyanobenzoic acid (147 mg, 1.0 mmol), following the procedure outlined in Example 1, gave 4-cyano-N-[2-(2,2,8-trimethyl-4H-[1,3]dioxino[4,5-c]pyridin-5-yl)-ethyl]-benzamide (58) (151 mg, 43% yield) as a colorless solid.

¹H-NMR (CDCl₃): δ 9.01 (s, 1H), 8.36 (s, 1H), 7.90 (d, 2H), 7.47 (d, 2H), 5.11 (s, 2H), 3.82 (m, 2H), 3.09 (s, 2H), 2.54 (s, 3H), 1.59 (s, 6H).

### Example 56

### Synthesis of 4-Carbamimidoyl-N-[2-(5-hydroxy-4-hydroxymethyl-6-methyl-pyridin-3-yl)-ethyl]-benzamide (59)

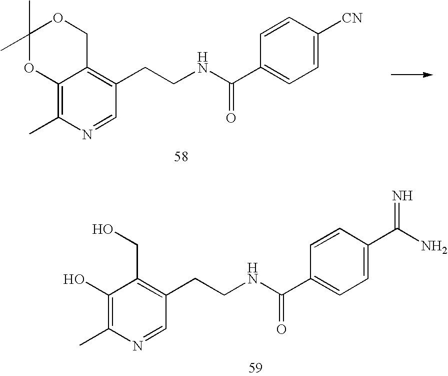

The conversion of nitrile (58) to amidine (59) was carried out as described in Example 2.

¹H-NMR (DMSO-d₆): δ 9.39-9.12 (m, 3H), 8.86 (s br, 1H), 8.04 (s, 1H), 7.99 (d, 2H), 7.88 (d, 2H), 3.55-3.48 (s br, 2H), 2.98 (t, 2H), 2.45 (s, 3H).

### Example 57

### Synthesis of 3-Cyano-N-[2-(2,2,8-trimethyl-4H-[1,3]dioxino[4,5-c]pyridin-5-yl)-ethyl]-benzamide (60)

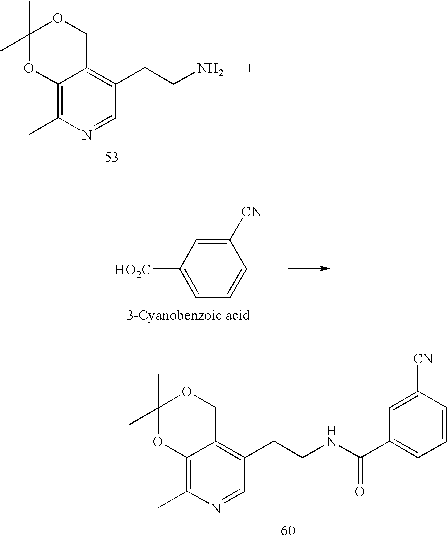

The coupling of 2-(2,2,8-trimethyl-4H-[1,3]dioxino[4,5-c]pyridin-5-yl)ethanamine (53) (771.4 mg, 3.47 mmol) and 3-cyanobenzoic acid (510 mg, 3.47 mmol), following the procedure described in Example 1, gave 3-cyano-N-[2-(2,2,8-trimethyl-4H-[1,3]dioxino[4,5-c]pyridin-5-yl)-ethyl]-benzamide (60) (850 mg, 70% yield) as a colorless solid.

¹H-NMR (CDCl₃): δ 9.00 (s br, 1H), 8.39 (s, 1H), 8.10 (s, 1H), 8.07 (m, 1H), 7.56 (m, 1H), 7.37 (m, 1H), 5.11 (s, 2H), 3.82 (m, 2H), 3.06 (t, 2H), 2.54 (s, 3H), 1.59 (s, 6H)

### Example 58

### Synthesis of 3-Carbamimidoyl-N-[2-(5-hydroxy-4-hydroxymethyl-6-methyl-pyridin-3-yl)-ethyl]-benzamide (61)

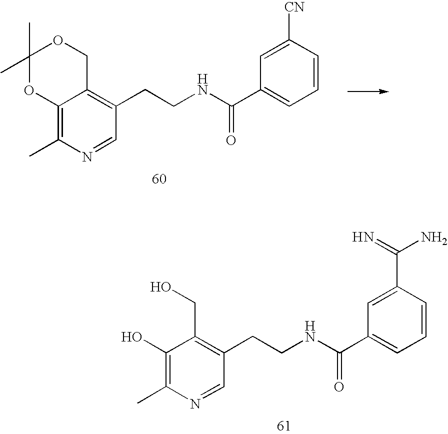

The conversion of nitrile (68) to amidine (69) was carried out as described in Example 2.

¹H-NMR (DMSO-d₆): δ 10.87 (s br, 1H), 9.55-9.26 (s, 3H), 9.05 (t, 1H), 8.41 (s, 1H), 8.21 (s, 1H), 8.13 (d, 1H), 7.97 (d, 1H), 7.70 (t, 1H), 4.85 (s, 2H), 3.57 (m, 2H), 3.10 (t, 2H), 2.57 (s, 3H).

### Example 59

### Inhibition of Platelet Aggregation

Platelet rich plasma (PRP) was obtained by drawing whole blood from normal human donors (not on any medication) into sodium citrate tubes (3.2%), and centrifuging at 160 xg for about 10 minutes. Platelet poor plasma (PPP) was obtained by centrifuging the remainder of the sample after the platelets were removed at 800 xg for about 10 minutes. The PRP was adjusted to a count of 280×10⁹/L using a mixture of PRP and PPP. The platelets (200 μL) were incubated with the test compounds (25 μL) adjusted to various concentrations (50, 100, 250, and 500 μM) for about 30 minutes at room temperature (approximate final platelet count in the incubation mixture of 250×10⁹/L). The samples were incubated for about 3 minutes at about 37° C., and then transferred to the mixing wells of a Chrono-log 4 channel aggregometer (Chrono-log Corp., Havertown, Pa.). After baselines were established, the agonist (25 μL of 40 μM ADP (Sigma, St. Louis, Mo.) or 25 μL of 50 μg/mL and 10 μg/mL collagen (Helena Laboratories, Beaumont, Tex.) or 25 μL of 120 μM thrombin receptor activating peptide (TRAP) (Sigma)) was then added. Aggregation was monitored for 5 minutes at 37° C. with stirring (1000 rpm). The amplitude and slope of each tracing were calculated to determine the amount of aggregation. Control samples were performed using only solvent. The % reduction in aggregation was calculated for each sample compared to the proper solvent control. See Table 1.

TABLE 1
Platelet inhibition

| | | % Reduction in Aggregation | | | |
|---|---|---|---|---|---|
| | Concentration | Collagen | Collagen | ADP | TRAP |
| Compound | (μM) | (5 μg/mL) | (1 μg/mL) | (4 μM) | (12 μM) |
| 2 | 500 | 12 | 3 | 10 | 6 |
| 5 | 500 | 27 | 91 | 71 | 84 |
| 7 | 500 | 8 | 2 | 26 | 34 |
| 9 | 500 | 10 | 0 | 2 | 0 |
| 14 | 500 | 3 | 0 | 16 | 8 |
| 55 | 500 | 20 | 24 | 38 | 75 |
| 57 | 500 | 0 | 6 | 0 | 14 |
| 59 | 500 | 11 | 28 | 55 | 67 |
| 61 | 500 | 10 | 0 | 2 | 0 |
| 18 | 250 | 7 | 6 | 10 | 30 |
| 20 | 250 | 64 | 94 | 93 | 95 |
| 23 | 250 | 17 | 19 | 39 | 27 |
| 25 | 250 | 29 | 81 | 63 | 30 |
| 28 | 250 | 24 | 17 | 46 | 10 |
| 31 | 250 | 28 | 74 | 79 | 64 |
| 35 | 250 | 12 | 86 | 84 | 35 |
| 37 | 250 | 6 | 19 | 43 | 17 |
| 43 | 250 | 0 | 100 | 87 | 69 |
| 26 | 100 | 18 | 52 | 62 | 17 |
| 39 | 100 | 8 | 1 | 3 | 5 |
| 52 | 50 | 7 | 93 | 95 | 95 |

1. A compound of the formula: 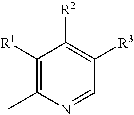
wherein
R¹ is OH, O-alkyl, or O-alkyl-aryl-R⁴, where R⁴ is H, —CN, amidine, alkyl, or cycloalkyl;
R² is alkyl; —(CH₂)_{n′}OH where n′ is an integer from 1 to 8; —(CH₂)ₙCOOH where n is an integer from 0 to 8; —(CH₂)ₙCOO(CH₂)ₙCH₃ where n is as defined above; (CH₂)ₙ-aryl-R⁵ where n is as defined above, and R⁵ is —CN or amidine; (CH₂)ₙ-aryl-aryl-R⁵, where n and R⁵ are as defined above; (CH₂)ₙ—NH-aryl-R⁵, where n and R⁵ are as defined above; (CH₂)ₙ—NH—CO-aryl-R⁴ where n and R⁴ are as defined above; (CH₂)ₙ—NH-aryl-aryl-R⁵ where n and R⁵ are as defined above; and (CH₂)ₙ—NH—CO-aryl-aryl-R⁶ where n is as defined above and R⁶ is —CN, —NO₂, NH₂, or amidine;
R³ is —(CH₂)_{n′}OH where n′ is as defined above; (CH₂)ₙ—NH-aryl-R⁵, where n and R⁵ are as defined above; (CH₂)ₙ—NH—CO-aryl-R⁴ where n and R⁴ are as defined above; (CH₂)ₙ—NH-aryl-aryl-R⁵ where n and R⁵ are as defined above; and (CH₂)ₙ—NH—CO-aryl-aryl-R⁶ where n and R⁶ are as defined above; and
R¹ and R² when taken together can form a compound of the formula 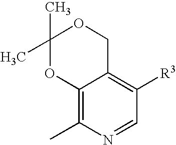
wherein R³ is as defined above, with the proviso that R³ cannot be CH₂—NH-Phenyl-R⁵ or CH₂—NH-Phenyl-Phenyl-R⁵; and
wherein only one of R⁴, R⁵, and R⁶ can be amidine; or a pharmaceutically acceptable salt thereof.

2. A pharmaceutical composition comprising a therapeutically effective amount of at least one compound of claim 1 and a pharmaceutically acceptable carrier.

3. The compound of claim 1, wherein an alkyl of 1-8 carbon atoms is unsubstituted or substituted with one or more of fluorine, chlorine, alkoxy groups of 1 to 8 carbon atoms, or amido groups having from 1 to 8 carbon atoms.

4. The compound of claim 3, wherein the alkoxy group is methoxy or ethoxy.

5. The compound of claim 3, wherein the amido group is acetamido.

6. The compound of claim 1, wherein an aryl group is a phenyl group or a naphthyl group.

7. The compound of claim 1, wherein an aryl group is substituted with one or more of fluorine, chlorine, bromine, alkyl groups having 1 to 8 carbon atoms, alkoxy groups having 1 to 8 carbon atoms, alkoxyalkyl groups having 1 to 8 carbon atoms and one or more oxygen atoms, or amido groups having 1 to 8 carbon atoms.

8. The compound of claim 7, wherein the alkyl group is methyl or ethyl.

9. The compound of claim 7, wherein the alkoxy group is methoxy or ethoxy.

10. The compound of claim 7, wherein the amido group is acetamido.

11. The compound of claim 1, wherein an aryl group is substituted with one or more functional groups.

12. The compound of claim 11, wherein the functional group is a hydroxy group, carboxy group, or acetoxy group.

13. A compound of the formula 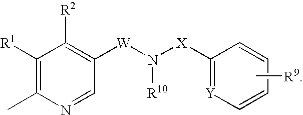
wherein
R¹ is OH, OCH₃, or OCH₂-(4-tert-butylphenyl);
R² is CH₂OH, CH₂OCH₃, CH₂OBn, CH₃, 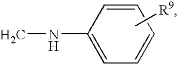
or COOR¹¹ where R¹¹ is H or alkyl;
W is (CH₂)_{n′} where n′=1, 2 or 3, or C═O;
X is (CH₂)ₙ where n=0, 1, 2, or 3, C═O, or CHCH₂CO₂H;
Y is C—H, C—F, C—OCH₃, C—OCF₃, C—CF₃, or N;
R⁹ is 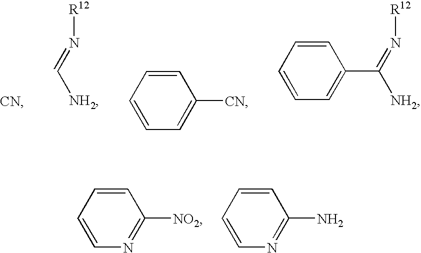
Where R¹² is H, OH or O-alkyl;
R¹⁰ is H, CH₂—Ar—R⁹ where R⁹ is defined as above;
R¹ and R² taken together can form a compound of the formula IV 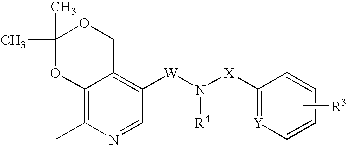
wherein W, X, Y, R³ and R⁴ are as described above; and
only one of R³ and R⁴ can be 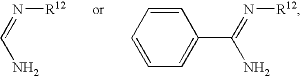
R¹² is as described above; or a pharmaceutically acceptable salt thereof.

14. The compound of claim 13, wherein the compound is 4-Carbamimidoyl-N-(5-hydroxyl-4-hydroxymethyl-6-methyl-pyridin-3-ylmethyl)-benzamide.

15. The compound of claim 13, wherein the compound is 4′-Carbamimidoyl-biphenyl-4-carboxylic (5-hydroxyl-4-hydroxymethyl-6-methyl-pyridin-3-ylmethyl)-amide.

16. The compound of claim 13, wherein the compound is 4′-[(5-Hydroxy-4,6-dimethyl-pyridin-3-ylmethyl)-amino]-biphenyl-4-carboxamidine.

17. The compound of claim 13, wherein the compound is 4-(N-(4-Carbamimidoyl-benzyl)-N-((5-hydroxy-4-(hydroxymethyl)-6-methylpyridin-3-yl)methyl)amino)benzamidine.

18. A method of treating cardiovascular, cerebro-vascular, or related diseases and symptoms in a mammal comprising administering a therapeutically effective amount of a compound according to claim 1.

19. The method of claim 18, wherein said compound is administered enterally, parenterally, or by inhalation.

20. A method of treating cardiovascular, cerebro-vascular, or related diseases and symptoms in a mammal comprising administering a therapeutically effective amount of a compound according to claim 13.

21. The method of claim 20, wherein the compound is 4-Carbamimidoyl-N-(5-hydroxyl-4-hydroxymethyl-6-methyl-pyridin-3-ylmethyl)-benzamide.

22. The method of claim 20, wherein the compound is 4′-Carbamimidoyl-biphenyl-4-carboxylic (5-hydroxyl-4-hydroxymethyl-6-methyl-pyridin-3-ylmethyl)-amide.

23. The method of claim 20, wherein the compound is 4′-[(5-Hydroxy-4,6-dimethyl-pyridin-3-ylmethyl)-amino]-biphenyl-4-carboxamidine.

24. The method of claim 20, wherein the compound is 4-(N-(4-Carbamimidoyl-benzyl)-N-((5-hydroxy-4-(hydroxymethyl)-6-methylpyridin-3-yl)methyl)amino)benzamidine.

25. The method of claim 19, wherein the compound is administered concurrently with another therapeutic agent.

26. A pharmaceutical composition comprising a therapeutically effective amount of a compound according to claim 13 and a pharmaceutically acceptable carrier.

27. A method of treating a mammal post-surgically comprising administering a therapeutically effective amount of a compound according to claim 1 following a surgical procedure.

28. A method of claim 27, wherein the surgical procedure is a hip replacement, angioplasty, or invasive cardiovascular surgery.

29. A method of claim 28, wherein the invasive cardiovascular surgery is coronary artery bypass graft or heart valve replacement.

30. A method of claim 28, wherein the angioplasty is coronary, pulmonary, peripheral, intracranial, extracranial carotid, renal, and aortic angioplasty.

31. The method of claim 27, wherein the compound is administered concurrently with another therapeutic agent.

32. The method of claim 27, wherein the compound is coated on a medical device.

33. The method of claim 32, wherein the medical device is an intravascular stent or catheter.

34. A method of treating a mammal post-surgically comprising administering a therapeutically effective amount of a compound according to claim 13 following a surgical procedure.

35. A method of claim 34, wherein the surgical procedure is a hip replacement, angioplasty, or invasive cardiovascular surgery.

36. A method of claim 35, wherein the invasive cardiovascular surgery is coronary artery bypass graft or heart valve replacement.

37. A method of claim 35, wherein the angioplasty is coronary, pulmonary, peripheral, intracranial, extracranial carotid, renal, and aortic angioplasty.

38. The method of claim 34, wherein the compound is administered concurrently with another therapeutic agent.

39. The method of claim 34, wherein the compound is coated on a medical device.

40. The method of claim 39, wherein the medical device is an intravascular stent or catheter.

41. The method of claim 34, wherein the compound is 4-Carbamimidoyl-N-(5-hydroxyl-4-hydroxymethyl-6-methyl-pyridin-3-ylmethyl)-benzamide.

42. The method of claim 34, wherein the compound is 4′-Carbamimidoyl-biphenyl-4-carboxylic (5-hydroxyl-4-hydroxymethyl-6-methyl-pyridin-3-ylmethyl)-amide.

43. The method of claim 34, wherein the compound is 4′-[(5-Hydroxy-4,6-dimethyl-pyridin-3-ylmethyl)-amino]-biphenyl-4-carboxamidine.

44. The method of claim 34, wherein the compound is 4-(N-(4-Carbamimidoyl-benzyl)-N-((5-hydroxy-4-(hydroxymethyl)-6-methylpyridin-3-yl)methyl)amino)benzamidine.

45. The method of claim 25, wherein said other therapeutic agent is an anti-platelet agent, glycoprotein IIb/IIIa inhibitor, or anticoagulant.

46. The method of claim 45, wherein said anti-platelet agent is clopidogrel, aspirin, or dipyridamole.

47. The method of claim 45, wherein said glycoprotein IIb/IIIa inhibitor is integrillin.

48. The method of claim 45, wherein said anticoagulant is unfractionated heparin, low molecular weigh heparins, hirudin, or argatroban.
